(19) United States
(12) Patent Application Publication
(10) Pub. No.: US 2026/0176680 A1
(43) Pub. Date: Jun. 25, 2026

(54) BIOCHEMICAL ANALYSIS SYSTEM AND METHOD OF CONTROLLING A BIOCHEMICAL ANALYSIS SYSTEM

(71) Applicant: Oxford Nanopore Technologies PLC, Oxford (GB)

(72) Inventors: John Beaulaurier, Oxford (GB); Sergey Aganezov, Oxford (GB)

(73) Assignee: Oxford Nanopore Technologies PLC, Oxford (GB)

(21) Appl. No.: 19/125,832

(22) PCT Filed: Oct. 25, 2023
(86) PCT No.: PCT/GB2023/052792

§ 371 Date: Apr. 30, 2025

Related U.S. Application Data
(60) Provisional application No. 63/421,325, filed on Nov. 1, 2022.

(51) Int. Cl. C12Q 1/6869; C12Q 1/6853; G06N 3/045; G06N 3/08; G06N 3/088; G06N 20/00; G06N 20/20

(52) U.S. Cl. CPC C12Q 1/6869; C12Q 1/6853; G06N 3/045; G06N 3/08; G06N 3/088; G06N 20/00; G06N 20/20

(57) ABSTRACT

A method of controlling a biochemical analysis system for analysing polymers comprising a sequence of polymer units is provided. The system is operable to take successive measurements of a polymer from a sensor element during translocation of the polymer with respect to a nanopore of the sensor element. The method comprises, when a polymer has partially translocated through the nanopore, analysing the measurements taken from the polymer during the partial translocation thereof to determine modification information in respect of a portion of the sequence of the polymer units. The polymer is classified as belonging to one of a set of classes based on the modification information; and the system is operated to reject the polymer or continue taking measurements from the polymer based on the class to which the polymer unit is classified as belonging.

### RELATED APPLICATIONS

This application is a national stage filing under 35 U.S.C. § 371 of international PCT application PCT/GB2023/052792, filed Oct. 25, 2023, which claims priority under 35 U.S.C. § 119(e) to U.S. provisional patent application, U.S. Ser. No. 63/421,325, filed Nov. 1, 2022, the entire contents of each of which are incorporated by reference herein.

### BACKGROUND

The present invention relates to identifying units of a polymer chain, more particularly to controlling an analysis system to identify a desired polymer chain.

There are many types of biochemical analysis system that provide measurements of polymer units for the purpose of determining the sequence. For example but without limitation, one type of measurement system uses a nanopore. Biochemical analysis systems that use a nanopore have been the subject of much recent development. Typically, successive measurements of a polymer are taken from a sensor element comprising a nanopore during translocation of the polymer through the nanopore. Some property of the system depends on the polymer units in the nanopore, and measurements of that property are taken. This type of measurement system using a nanopore has considerable promise, particularly in the field of sequencing a polynucleotide such as DNA or RNA.

Such biochemical analysis systems using nanopores can provide long continuous reads of polymers, for example in the case of polynucleotides ranging from many hundreds to tens of thousands (and potentially more) nucleotides. The data gathered in this way comprises measurements, such as measurements of ion current, where each translocation of the sequence through the sensitive part of the nanopore results in a slight change in the measured property. Whilst such biochemical analysis systems using nanopores can provide significant advantages, it remains desirable to increase the speed of analysis.

### SUMMARY

According to an aspect of the present invention, there is provided a method of controlling a biochemical analysis system for analysing polymers that comprise a sequence of polymer units, wherein the biochemical analysis system comprises at least one sensor element that comprises a nanopore, and the biochemical analysis system is operable to take successive measurements of a polymer from the sensor element during translocation of the polymer with respect to the nanopore of the sensor element, the method comprising: when a polymer has partially translocated through the nanopore, analysing the measurements taken from the polymer during the partial translocation thereof to determine modification information in respect of a portion of the sequence of the polymer units, the modification information representing a sequence of estimates of modification statuses of subject polymer units of the portion of modification with respect to at least one canonical type of polymer unit; classifying the polymer as belonging to one of a set of classes based on the modification information; and operating the biochemical analysis system to reject the polymer or continue taking measurements from the polymer based on the class to which the polymer unit is classified as belonging.

The present invention analyses real-time measurements of polymers translocating a nanopore (e.g., translocation of a DNA/RNA molecule) and makes a decision on whether to continue sequencing the currently translocating polymer or to issue a signal to reject the polymer in question from the nanopore based on its modification properties. Rejection of polymers that are not of interest allows the nanopore to spend the (overall limited) polymer-translocating time on other input molecules. This results in the nanopore translocating (e.g., sequencing) more of a desired subset of the input molecules based on the desire to enrich/deplete a particular subset of the whole set of molecules loaded onto the nanopore-based biochemical analysis system. Modification status of polymer units can be indicative of a range of properties of interest, such as the biological origin of the polymer, and so is a useful classification on which to be able to choose to reject the polymer.

In some embodiments, the estimates of modification statuses comprise scores in respect of the subject polymer units. The use of scores allows the method to account for a level of certainty in the estimate of the modification status.

In some embodiments, the method further comprises determining sequence information representing estimates of the identities of the polymer units of the portion of the sequence of polymer units. Identifying the polymer units can provide further information to inform the determination of the modification status of the polymer units.

In some embodiments, the estimates of the identities of the polymer units comprise scores in respect of each of a set of types of polymer units. The use of scores allows the method to account for a level of certainty in the estimate of the identity of the polymer units.

In some embodiments, the estimates of the identities of the polymer units are estimates in respect of a set comprising canonical types of polymer units, and determining the modification information comprises analysing the sequence information to determine the modification information. First determining the sequence information as canonical types of polymer units and then using the sequence information to determine modification information reduces the number of categories into which the polymer units must be classified at each stage. This can improve the overall accuracy of identifying the polymer units, for example when using machine learning techniques.

In some embodiments, the estimates of the identities of the polymer units are estimates in respect of a set comprising canonical types of polymer units and one or more modified forms of at least one canonical type of the polymer units, wherein the modification information comprises the estimates in respect of the modified forms the at least one canonical type of the polymer units. Determining the full identity of the polymer units in a single step reduces the complexity of the classification process, which may be advantageous in increased speed of classification.

In some embodiments, the polymer is classified as belonging to one of the set of classes based on the modification information and the sequence information. Using both types of information can increase the amount of data available on which to base classification, thereby improving its accuracy.

In some embodiments, the polymer is classified as belonging to one of the set of classes based on the modification information only. Using only the modification information reduces the complexity of the classification process, which can provide advantageous in increased speed and reduced memory requirements.

In some embodiments, the polymer derives from an organism, and wherein the classes of the set of classes are taxonomic domains or kingdoms. In some embodiments, a first class of the set of classes is bacterial organisms or a type of bacterial organism, and a second class of the set of classes is eukaryotic organisms or a type of eukaryotic organism. These divisions allow the method to differentiate common categories of polymer origin that may be of interest for sequencing from categories that may not be of interest.

In some embodiments, at least one class of the set of classes represents a target sequence, and the step operating the biochemical analysis system to reject the polymer or continue taking measurements from the polymer comprises operating the biochemical analysis system to continue taking measurements when the class to which the polymer unit is classified as belonging by the machine learning classifier being said at least one class representing a target sequence. This allows the system to continue to sequence the polymer when it is determined to be of a type that is of interest.

In some embodiments, at least one class of the set of classes represents a background sequence, and the step operating the biochemical analysis system to reject the polymer or continue taking measurements from the polymer comprises operating the biochemical analysis system to reject the polymer when the class to which the polymer unit is classified as belonging by the machine learning classifier being said at least one class representing a background sequence. This allows the system to reject the polymer (thereby freeing the nanopore for sequencing of other polymers) when the polymer is determined to be of a type not of interest, or representing a background contaminant.

In some embodiments, the polymer is a polynucleotide, and the polymer units are nucleotides. Sequencing of polynucleotides is of particular interest for many biological applications.

In some embodiments, the modification statuses are methylation statuses. In some embodiments, the subject polymer units are cytosine nucleotides and the methylation statuses are statuses of methylation to at least one of 5-methyl-cytosine or 5-hydroxymethyl-cytosine; and/or the subject polymer units are adenosine nucleotides and the methylation statuses are statuses of methylation to 6-methyl-adenine. Methylation status, in particular of cytosine and/or adenosine, is a common modification to nucleotides that can be indicative of a wide range of biological factors such as disease or biological origin, and is therefore of particular interest for the classes.

In some embodiments, the modification statuses are oxidation statuses. Oxidation status can also be indicative of changes to a polymer that may be of interest for classifying and selecting the polymers to be sequenced.

In some embodiments, the subject polymer units comprise polymer units forming part of a predetermined motif of polymer units. Particular combinations of polymer units may be commonly found together or be particularly useful as indicators of class. Therefore choosing a motif of polymer units as the subject polymer units can improve accuracy of the classification by relying on these combinations of polymer units.

In some embodiments, the predetermined motif of polymer units is a cytosine nucleotide followed by a guanine nucleotide in the sequence of nucleotides along a 5′→3′ direction. The modification status of this particular combination of nucleotides can be highly predictive of classes such as biological origin, and is therefore a useful choice of motif.

In some embodiments, the at least one sensor element is operable to eject a polymer that is translocating through the nanopore, wherein operating the biochemical analysis system to reject the polymer comprises operating the sensor element to eject the polymer from the nanopore and accept a further polymer in the nanopore. Ejecting the polymer frees up the nanopore for sequencing of another molecule of interest, thereby speeding up the sequencing of the desired class of polymers.

In some embodiments, the at least one sensor element is operable to eject a polymer that is translocating through the nanopore by application of an ejection bias voltage sufficient to eject the polymer, wherein operating the sensor element to eject the polymer from the nanopore is performed by applying an ejection bias voltage. Using a bias voltage can be effective in ejecting polymers due to their static charge. A bias voltage is also often used to encourage polymers to enter the nanopore, and so reversal of the voltage is a convenient way to provide ejection functionality without the requirement for substantial hardware modifications.

In some embodiments, classifying the polymer as belonging to one of the set of classes comprises inputting the modification information into a machine learning classifier that classifies the polymer as belonging to one of the set of classes based on the modification information. Using a machine learning classifier allows the classification to more accurately classify previously unseen polymers or variations of polymers, compared to other techniques such as comparison to a database of reference polymer sequences. In some embodiments, the machine learning classifier comprises a neural network. Neural networks have been found to be effective for applications of this type.

In some embodiments, the machine learning classifier is trained using modification information in respect of plural classes. Using information from plural classes increases the contrast in the training data available to the machine learning classifier, thereby improving its ability to distinguish the plural classes and accurately identify the correct class for a particular polymer.

The invention also provides a computer program comprising instructions which, when the program is executed by a computer, cause the computer to carry out a method according to the above aspect or any embodiment thereof. There is also provided a computer storage medium storing said computer program.

There is also provided a biochemical analysis system for analysing polymers that comprise a sequence of polymer units, the biochemical analysis system comprising at least one sensor element that comprises a nanopore, wherein the biochemical analysis system is operable to take successive measurements of a polymer from the sensor element during translocation of the polymer with respect to the nanopore of the sensor element; and wherein the biochemical analysis system is configured to perform the method of the above aspect or any embodiment thereof. The biochemical analysis system may be a portable biochemical analysis system.

### BRIEF DESCRIPTION OF DRAWINGS

To allow better understanding, embodiments of the present invention will now be described by way of non-limitative example with reference to the accompanying drawings, in which:

FIG. 1 is a schematic diagram of a biochemical analysis system;

FIG. 2 is a plot of a typical measurement signal over time;

FIG. 3 is a schematic of a single sensor element comprising a nanopore;

FIG. 4 is a flowchart of a method of controlling the biochemical analysis system;

FIG. 5 is a flowchart of a method of determining modification information, which may be used in the analysis step of FIG. 4;

FIG. 6 is a flowchart showing further detail of analysing the sequence information in the method of FIG. 5;

FIG. 7 illustrates generating sequence slices for use in determining modification information;

FIG. 8 illustrates the use of sequence slices by a neural network;

FIG. 9 is a flowchart of an alternative method of determining modification information to the method of FIG. 5;

FIG. 10 shows a comparison of computational performance of input data preprocessing steps on different read lengths and batch sizes when performed on a CPU and a GPU;

FIG. 11 shows a comparison of computational performance of input data classification on different read lengths and batch sizes when performed on a CPU and a GPU;

FIG. 12 shows an exemplary neural network architecture for classifying the polymer;

FIG. 13 shows results illustrating the performance of the exemplary classifier in distinguishing DNA of plant and vertebrate origin based only on modification information;

FIG. 14 shows results illustrating the performance of the exemplary classifier in distinguishing DNA from a range of origins based only on modification information;

FIG. 15 shows results illustrating the performance of the exemplary classifier in distinguishing DNA of plant and vertebrate origin based on modification information and sequence information;

FIG. 16 shows results illustrating the performance of the exemplary classifier in distinguishing DNA from a range of origins based on modification information and sequence information;

FIG. 17 shows differences for a first set of channels in distributions of read lengths for DNA of human and bacterial origin with and without the use of the present method; and

FIG. 18 shows differences for a second set of channels in distributions of read lengths for DNA of human and bacterial origin with and without the use of the present method.

### DETAILED DESCRIPTION

The present invention concerns a method of controlling a biochemical analysis system. The biochemical analysis system may be a biochemical analysis system as described in WO2016/059427A1, which is incorporated herein by reference. The biochemical analysis system may be a portable biochemical analysis system.

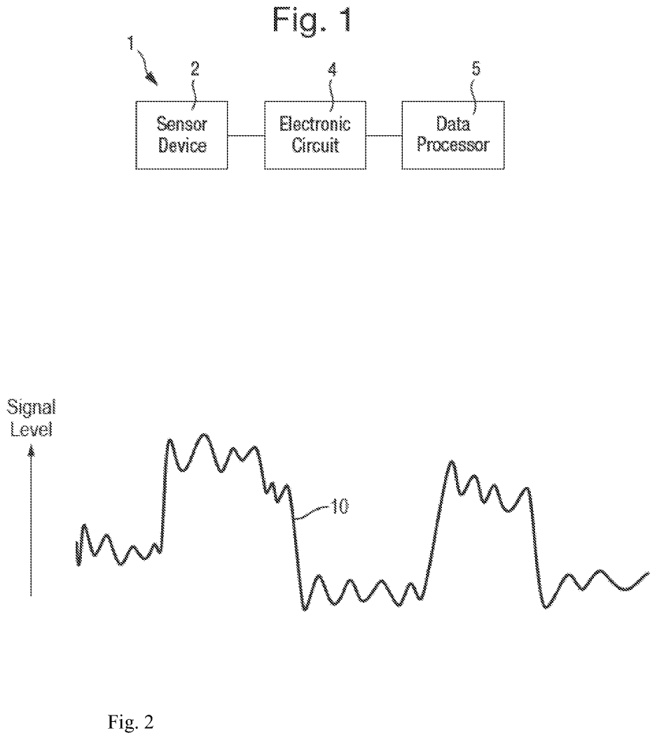

FIG. 1 is a schematic illustration of a biochemical analysis system 1 that may be controlled using the present method. The biochemical analysis system 1 is for analysing polymers, and may also be used for sorting polymers. Reverting to FIG. 1, the biochemical analysis system 1 comprises a sensor device 2 connected to an electronic circuit 4, which is in turn connected to a data processor 6.

The biochemical analysis system 1 comprises at least one sensor element, as discussed further below. The at least one sensor element may be comprised in the sensor device 2. The sensor element comprises a nanopore, and the biochemical analysis system 1 is operable to take successive measurements of a polymer from the sensor element during translocation of the polymer with respect to the nanopore of the sensor element. The polymer comprises a sequence (or series) of polymer units. The sensor device 2 derives a measurement signal 10 from the polymer, for example comprising the successive measurements. The successive measurements may be referred to as the measurement signal 10 below. The data processor 5 performs analysis of the measurement signal 10 to derive information about the polymer.

In some preferred applications, the polymer is a polynucleotide (or nucleic acid), and the polymer units are nucleotides. However, in general the polymer may be of any type, for example a polypeptide such as a protein, or a polysaccharide. The polymer may be natural or synthetic. The polynucleotide may comprise a homopolymer region. The homopolymer region may comprise between 5 and 15 nucleotides.

In the case of a polynucleotide or nucleic acid, the polymer units are nucleotides. The nucleic acid is typically deoxyribonucleic acid (DNA), ribonucleic acid (RNA), or a synthetic nucleic acid known in the art, such as peptide nucleic acid (PNA), glycerol nucleic acid (GNA), threose nucleic acid (TNA), locked nucleic acid (LNA) or other synthetic polymers with nucleotide side chains. The PNA backbone is composed of repeating N-(2-aminoethyl)-glycine units linked by peptide bonds. The GNA backbone is composed of repeating glycol units linked by phosphodiester bonds. The TNA backbone is composed of repeating threose sugars linked together by phosphodiester bonds. LNA is formed from ribonucleotides as discussed above having an extra bridge connecting the 2′ oxygen and 4′ carbon in the ribose moiety. The nucleic acid may be single-stranded, be double-stranded or comprise both single-stranded and double-stranded regions. The nucleic acid may comprise one strand of RNA hybridised to one strand of DNA. Typically cDNA, RNA, GNA, TNA or LNA are single stranded.

The polymer units may be any type of nucleotide. The nucleotide can be naturally occurring or artificial. For instance, the method may be used to verify the sequence of a manufactured oligonucleotide. A nucleotide typically contains a nucleobase, a sugar and at least one phosphate group. The nucleobase and sugar form a nucleoside. The nucleobase is specifically adenine, guanine, thymine, uracil and cytosine. The sugar is typically a pentose sugar. Suitable sugars include, but are not limited to, ribose and deoxyribose. The nucleotide is typically a ribonucleotide or deoxyribonucleotide. The nucleotide typically contains a monophosphate, diphosphate or triphosphate.

The polymer units may be canonical polymer units. For example in the case that the polymer is a DNA polynucleotide, the canonical bases are adenine (A), cytosine (C), guanine (G), and thymine (T). By contrast ribonucleic acid (RNA) comprises the canonical bases A, C and G, with uracil (U) in place of thymine.

The nucleotide can be a modified polymer unit, such as a damaged or epigenetic base. For instance, the nucleotide may comprise a pyrimidine dimer. Such dimers are typically associated with damage by ultraviolet light and are the primary cause of skin melanomas. The nucleotide can be labelled or modified to act as a marker with a distinct signal. This technique can be used to identify the absence of a base, for example, an abasic unit or spacer in the polynucleotide. The method could also be applied to any type of polymer.

In the case of a polypeptide, the polymer units may be amino acids that are naturally occurring or synthetic. In the case of a polysaccharide, the polymer units may be monosaccharides.

Particularly where the sensor device 2 comprises a nanopore and the polymer comprises a polynucleotide, the polynucleotide under investigation may range in length from typically 500 nucleotides (500b) to greater than 2 Mb in length. However polynucleotides of shorter length may be measured with the lower limit estimated to be around 10-20 bases depending upon the length of the nanopore channel, which would include mRNA, tRNA and cfDNA.

Where the polymer comprises a polynucleotide, the polynucleotide is preferably a native polynucleotide molecule, i.e. a polynucleotide molecule that has not been processed using amplification techniques such as polymerase chain reaction (PCR). This is because amplification process such as PCR may remove or obscure chemical modifications of the nucleotide bases. However, the method may be used with polymers that have undergone amplification processes if the amplification is such as to allow preservation of the modification status of the polymer units.

The nature of an example sensor device 2 and a resultant measurement signal 10 is as follows.

The sensor device 2 is a nanopore system that comprises one or more nanopores. In a simple type, the sensor device 2 has only a single nanopore, but a more practical measurement systems employ many nanopores, typically in an array, to provide parallelised collection of information. The measurement system may comprise at least 10 nanopores, optionally at least 100 nanopores, optionally at least 1000 nanopores.

The measurement signal 10 may be recorded during translocation of the polymer with respect to the nanopore, typically through the nanopore.

The nanopore is a pore, typically having a size of the order of nanometres, which may allow the passage of polymers therethrough. The nanopore may be a protein pore or a solid state pore. The dimensions of the pore may be such that only one polymer may translocate the pore at a time.

Where the nanopore is a protein pore, it may have the following properties.

The biological pore may be a transmembrane protein pore. Transmembrane protein pores for use in accordance with the invention can be derived from β-barrel pores or α-helix bundle pores. β-barrel pores comprise a barrel or channel that is formed from β-strands. Suitable β-barrel pores include, but are not limited to, β-toxins, such as α-hemolysin, anthrax toxin and leukocidins, and outer membrane proteins/porins of bacteria, such as Mycobacterium smegmatis porin (Msp), for example MspA, MspB, MspC or MspD, lysenin, outer membrane porin F (OmpF), outer membrane porin G (OmpG), outer membrane phospholipase A and Neisseria autotransporter lipoprotein (NalP). α-helix bundle pores comprise a barrel or channel that is formed from α-helices. Suitable α-helix bundle pores include, but are not limited to, inner membrane proteins and α outer membrane proteins, such as WZA and ClyA toxin. The transmembrane pore may be derived from Msp or from α-hemolysin (α-HL). The transmembrane pore may be derived from lysenin. Suitable pores derived from lysenin are disclosed in WO 2013/153359. Suitable pores derived from MspA are disclosed in WO-2012/107778. The pore may be derived from CsgG, such as disclosed in WO-2016/034591 and WO2019/002893, both herein incorporated by reference in their entirety. The pore may be a DNA origami pore.

The protein pore may be a naturally occurring pore or may be a mutant pore.

The protein pore may be inserted into an amphiphilic layer such as a biological membrane, for example a lipid bilayer. An amphiphilic layer is a layer formed from amphiphilic molecules, such as phospholipids, which have both hydrophilic and lipophilic properties. The amphiphilic layer may be a monolayer or a bilayer. The amphiphilic layer may be a co-block polymer such as disclosed in Gonzalez-Perez et al., Langmuir, 2009, 25, 10447-10450, WO2014/064444, or U.S. Pat. No. 6,723,814 herein incorporated by reference in its entirety. Alternatively, a protein pore may be inserted into an aperture provided in a solid state layer, for example as disclosed in WO2012/005857.

A suitable apparatus for providing an array of nanopores is disclosed in WO-2014/064443. The nanopores may be provided across respective wells wherein electrodes are provided in each respective well in electrical connection with an ASIC for measuring current flow through each nanopore. A suitable current measuring apparatus may comprise the current sensing circuit as disclosed in WO-2016/181118.

The nanopore may comprise an aperture formed in a solid state layer, which may be referred to as a solid state pore. The aperture may be a well, gap, channel, trench or slit provided in the solid state layer along or into which analyte may pass. Such a solid-state layer is not of biological origin. In other words, a solid state layer is not derived from or isolated from a biological environment such as an organism or cell, or a synthetically manufactured version of a biologically available structure. Solid state layers can be formed from both organic and inorganic materials including, but not limited to, microelectronic materials, insulating materials such as Si3N4, Al203, and SiO, organic and inorganic polymers such as polyamide, plastics such as Teflon® or elastomers such as two-component addition-cure silicone rubber, and glasses. The solid state layer may be formed from graphene. Suitable graphene layers are disclosed in WO-2009/035647, WO-2011/046706 or WO-2012/138357. Suitable methods to prepare an array of solid state pores is disclosed in WO-2016/187519.

Such a solid state pore is typically an aperture in a solid state layer. The aperture may be modified, chemically, or otherwise, to enhance its properties as a nanopore. A solid state pore may be used in combination with additional components which provide an alternative or additional measurement of the polymer such as tunnelling electrodes (Ivanov A P et al., Nano Lett. 2011 Jan. 12; 11(1):279-85), or a field effect transistor (FET) device (as disclosed for example in WO-2005/124888). Solid state pores may be formed by known processes including for example those described in WO-00/79257.

The nanopore may be a hybrid of a solid state pore with a protein pore.

The sensor device 2 takes a series of measurements of a property that depends on the polymer units translocating with respect to the pore may be measured. The series of measurements form the measurement signal 10.

The property that is measured may be associated with an interaction between the polymer and the pore. Such an interaction may occur at a constricted region of the pore.

In one type of sensor device 2, property that is measured may be the ion current flowing through a nanopore. These and other electrical properties may be measured using standard single channel recording equipment as describe in Stoddart D et al., Proc Natl Acad Sci, 12; 106(19):7702-7, Lieberman K R et al, J Am Chem Soc. 2010; 132(50):17961-72, and WO-2000/28312. Alternatively, measurements of electrical properties may be made using a multi-channel system, for example as described in WO-2009/077734, WO-2011/067559 or WO-2014/064443.

Ionic solutions may be provided on either side of the membrane or solid state layer, which ionic solutions may be present in respective compartments. A sample containing the polymer analyte of interest may be added to one side of the membrane and allowed to move with respect to the nanopore, for example under a potential difference or chemical gradient. The measurement signal 10 may be derived during the movement of the polymer with respect to the pore, for example taken during translocation of the polymer through the nanopore. The polymer may partially translocate the nanopore.

In order to allow measurements to be taken as the polymer translocates through a nanopore, the rate of translocation can be controlled by a polymer binding moiety. Typically the moiety can move the polymer through the nanopore with or against an applied field. The moiety can be a molecular motor using for example, in the case where the moiety is an enzyme, enzymatic activity, or as a molecular brake. Where the polymer is a polynucleotide there are a number of methods proposed for controlling the rate of translocation including use of polynucleotide binding enzymes. Suitable enzymes for controlling the rate of translocation of polynucleotides include, but are not limited to, polymerases, helicases, exonucleases, single stranded and double stranded binding proteins, and topoisomerases, such as gyrases. For other polymer types, moieties that interact with that polymer type can be used. The polymer interacting moiety may be any disclosed in WO-2010/086603, WO-2012/107778, and Lieberman K R et al, J Am Chem Soc. 2010; 132(50):17961-72), and for voltage gated schemes (Luan B et al., Phys Rev Lett. 2010; 104(23):238103). The rate of translocation of the polymer through the nanopore may be controlled by a voltage control pulse to step the polymer through the nanopore such as disclosed in WO2019/006214. Translocation of the polymer may be controlled by a molecular hopper such as disclosed by WO2020/016573.

The polymer binding moiety can be used in a number of ways to control the polymer motion. The moiety can move the polymer through the nanopore with or against the applied field. The polynucleotide binding enzyme does not need to display enzymatic activity as long as it is capable of binding the target polynucleotide and controlling its movement through the pore. For instance, the enzyme may be modified to remove its enzymatic activity or may be used under conditions which prevent it from acting as an enzyme. Such conditions are discussed in more detail below.

The polynucleotide binding enzyme may be a Dda helicase such as disclosed in WO2015055981, hereby incorporated by reference in its entirety.

Translocation of the polymer through the nanopore may occur, either cis to trans or trans to cis, either with or against an applied potential, applied by the electronic circuit 4 discussed below. The translocation may occur under an applied potential which may control the translocation. The binding enzyme is typically held against the cis or trans opening of the nanopore during translocation of the polynucleotide through the nanopore under an applied potential.

Exonucleases that act progressively or processively on double stranded DNA can be used on the cis side of the pore to feed the remaining single strand through under an applied potential or the trans side under a reverse potential. Likewise, a helicase that unwinds the double stranded DNA can also be used in a similar manner. There are also possibilities for sequencing applications that require strand translocation against an applied potential, but the DNA must be first “caught” by the enzyme under a reverse or no potential. With the potential then switched back following binding the strand will pass cis to trans through the pore and be held in an extended conformation by the current flow. The single strand DNA exonucleases or single strand DNA dependent polymerases can act as molecular motors to pull the recently translocated single strand back through the pore in a controlled stepwise manner, trans to cis, against the applied potential. Alternatively, the single strand DNA dependent polymerases can act as a molecular brake slowing down the movement of a polynucleotide through the pore. Any moieties, techniques or enzymes described in WO-2012/107778 or WO-2012/033524 could be used to control polymer motion.

However, the sensor device 2 may be of alternative types that comprise one or more nanopores.

Similarly, the properties that are measured may be of types other than ion current. Some examples of alternative types of property include without limitation: electrical properties and optical properties. A suitable optical method involving the measurement of fluorescence is disclosed by J. Am. Chem. Soc. 2009, 131 1652-1653. Possible electrical properties include: ionic current, impedance, a tunnelling property, for example tunnelling current (for example as disclosed in Ivanov A P et al., Nano Lett. 2011 Jan. 12; 11(1):279-85), and a FET (field effect transistor) voltage (for example as disclosed in WO2005/124888). One or more optical properties may be used, optionally combined with electrical properties (Soni G V et al., Rev Sci Instrum. 2010 January; 81(1):014301). The property may be a transmembrane current, such as ion current flow through a nanopore. The ion current may typically be the DC ion current, although in principle an alternative is to use the AC current flow (i.e. the magnitude of the AC current flowing under application of an AC voltage).

In some types of the sensor device 2, the measurement signal 10 may be characterised as comprising measurements from a series of events, where each event provides a group of measurements. FIG. 2 illustrates a typical example of such a measurement signal 10 in the case of measurement of current. The group of measurements from each event have a level that is similar, although subject to some variance. This may be thought of as a noisy step wave with each step corresponding to an event. The events may have biochemical significance, for example arising from a given state or interaction of the sensor device 2. This may in some instances arise from translocation of the polymer through the nanopore occurring in a ratcheted manner. However, this type of signal is not produced by all types of measurement system and the methods described herein are not dependent on the type of signal. For example, when translocation rates approach the measurement sampling rate, for example, measurements are taken at 1 times, 2 times, 5 times or 10 times the translocation rate of a polymer unit, events may be less evident or not present, compared to slower sequencing speeds or faster sampling rates.

In addition, where events are present, typically there is no a priori knowledge of number of measurements in the group, which varies unpredictably. These factors of variance and lack of knowledge of the number of measurements can make it hard to distinguish some of the groups, for example where the group is short and/or the levels of the measurements of two successive groups are close to one another.

The group of measurements corresponding to each event typically has a level that is consistent over the time scale of the event, but for most types of the sensor device 2 will be subject to variance over a short time scale. Such variance can result from measurement noise, for example arising from the electrical circuits and signal processing, notably from the amplifier in the particular case of electrophysiology. Such measurement noise is inevitable due the small magnitude of the properties being measured. Such variance can also result from inherent variation or spread in the underlying physical or biological system of the sensor device 2, for example a change in interaction, which might be caused by a conformational change of the polymer.

Most types of the sensor device 2 will experience such inherent variation to greater or lesser extents. For any given types of the sensor device 2, both sources of variation may contribute or one of these noise sources may be dominant.

With increase in the sequencing rate, being the rate at which polymer units translocate with respect to the nanopore, then the events may become less pronounced and hence harder to identify, or may disappear. Thus, analysis methods that rely on detecting such events detection may become less efficient at as the sequencing rate increases.

However, the methods disclosed herein are not dependent on detecting such events. The methods described below are effective even at relatively high sequencing rates, including sequencing rates at which the polymer translocates at a rate of at least 10 polymer units per second, preferably 100 polymer units per second, more preferably 500 polymer units per second, or more preferably 1000 polymer units per second.

The sample rate is the rate of measurements in the signal. Typically, the sample rate is higher than the sequencing rate. For example, the sample rate may be in a range from a 100 Hz to 30 kHz, but this is not limitative. In practice the sample rate may depend on the nature of the sensor device 2.

Reverting to FIG. 1, the electronic circuit 4 will now be discussed. The electronic circuit may be the electronic circuit 4 disclosed in WO2016059427, incorporated herein by reference in its entirety. The electronic circuit 4 is arranged to control the application of bias voltages across each sensor element of the sensor device 2. During normal operation, the bias voltage is selected to enable translocation of a polymer through the pore of a sensor element. Such a bias voltage may typically be of a level up to −200 mV.

The bias voltage supplied by the electronic circuit 4 may also be selected so that it is sufficient to eject the translocating polymer from the pore. By causing the electronic circuit 4 to supply such a bias voltage, the sensor element is operable to eject a polymer that is translocating through the pore. To ensure reliable ejection, the bias voltage is typically a reverse bias, although that is not always essential.

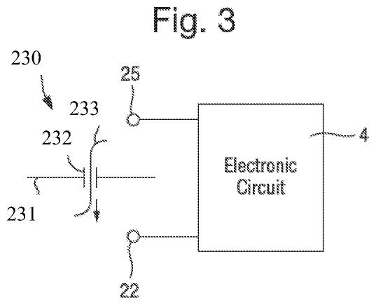

An example arrangement of an electronic circuit 4 is shown in FIG. 3. FIG. 3 illustrates a single sensor element 230 of the sensor device 2, showing an example polymer 233 translocating through a pore 232. The sensor element 230 is made by forming a membrane 231 across a respective well of the sensor device 2, and then by inserting a pore 232 into the membrane 31. The membrane 231 seals a respective well from a sample chamber of the sensor device 2.

The electronic circuit 4 is connected to electrodes 22, 25, connected on either side of the membrane 231. The electrode 25 may be a common electrode 25, common to multiple sensor elements 230. The electrode 22 may be a sensor electrode 25 for the respective sensor element 230. The electronic circuit 4 is controls the application of bias voltages to generate a bias between the electrodes 22, 25, to control translocation of the polymer 233 as described above. The sensor electrode 25 may be used to take electrical measurements as the polymer 233 translocates through the pore 232, which may be used as or processed to form the measurement signal 10.

Returning to FIG. 1, the data processor 5 connected to the electronic circuit 4 is arranged as follows. The data processor 5 may be a computer apparatus running an appropriate program, may be implemented by a dedicated hardware device, or may be implemented by any combination thereof. The computer apparatus, where used, may be any type of computer system but is typically of conventional construction. The computer program may be written in any suitable programming language. The computer program may be stored on a computer-readable storage medium, which may be of any type, for example: a recording medium which is insertable into a drive of the computing system and which may store information magnetically, optically or opto-magnetically; a fixed recording medium of the computer system such as a hard drive; or a computer memory. The data processor 5 may comprise a card to be plugged into a computer such as a desktop or laptop. The data used by the data processor 5 may be stored in a memory thereof in a conventional manner.

The data processor 5 receives and processes the successive measurements taken using the sensor element 230 of the sensor device 2. That is, the data processor 5 receives the measurement signal 10. The data processor 5 stores and analyses the successive measurements, as described further below.

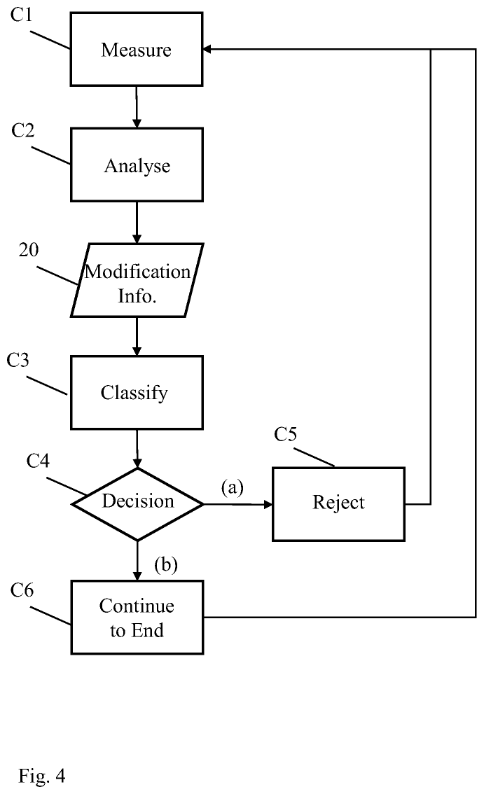

There will now be described a method shown in FIG. 4 of controlling the biochemical analysis system 1 to analyse polymers.

A biochemical analysis system, such as the biochemical analysis system 1 described above, may be configured to perform the method of FIG. 4. Alternatively a computer program may be provided comprising instructions which, when the program is executed by a computer or processor, cause the computer to carry out the method of FIG. 4. The computer program or instructions may be stored in a memory of a biochemical analysis system, such as the biochemical analysis system 1 described above. The processor may be a processor of a biochemical analysis system, such as the biochemical analysis system 1 described above. The computer program may be part of, or interact with, the firmware of the biochemical analysis system. Alternatively, the computer program may form a downstream component of the biochemical analysis system that performs the analysis and classification steps, and operates the biochemical analysis system by communicating the desired decision back to the hardware/firmware of the biochemical analysis system.

Alternatively a transitory or non-transitory computer readable medium may be provided comprising instructions which, when executed by a processor, cause the processor to carry out a method of FIG. 4. The processor may be a processor of a biochemical analysis system, such as the biochemical analysis system 1 described above. In some examples, the method is implemented in the data processor 5 of the biochemical analysis system 1 described above. This method may be performed in parallel in respect of each sensor element 230 from which successive measurements of a polymer are taken.

In step C1, the biochemical analysis system 1 is operated to apply a bias voltage across the pore of a sensor element 230 that is sufficient to enable translocation of polymer, for example using electronic circuit 4. Based on an output signal detected for example using the electronic circuit 4, translocation is detected and a measurement signal 10 starts to be taken. A series of successive measurements is taken over time.

In some cases, the following method steps operate on the series of raw measurements taken by the sensor device 2. In other cases, the raw measurements are pre-processed to derive a series of measurements that are used in the following method steps instead of the raw measurements.

Method step C2 is performed when a polymer has partially translocated through the nanopore, i.e. during the translocation. At this time, the series of measurements taken from the polymer during the partial translocation is collected for analysis, and the method comprises analysing C2 the measurements taken from the polymer during the partial translocation thereof to determine modification information 20 in respect of a portion of the sequence of the polymer units. The measurements taken from the polymer during the partial translocation may be referred to herein as a “chunk” of measurements.

Method step C2 may be performed after a predetermined number of measurements have been taken so that the chunk of measurements is of predefined size, for example corresponding to at most 100 polymer units, optionally at most 500 polymer units, optionally at most 1000 polymer units, optionally at most 5000 polymer units. Alternatively, method step may be performed after a predetermined amount of time has elapsed after the polymer begins translocating the nanopore, for example at most 10 seconds after the polymer begins translocating the nanopore, optionally at most 5 seconds, optionally at most 2 seconds, optionally at most 1 second, optionally at most 0.5 seconds, optionally at most 0.3 seconds, optionally at most 0.2 seconds, optionally at most 0.1 seconds. In the former case, the size of the chunk of measurements may be defined by parameters that are initialised at the start of a run (e.g. the start of a process of sequencing polymers within a sample), but are changed dynamically so that the size of the chunk of measurements changes. The size of the chunk of measurements may be chosen based on any suitable factors, for example a size required to be able to reliably classify the polymer as described in more detail below. The size of the chunk may vary depending on the application. Some applications may require a larger chunk so that a larger portion of the polymer can be considered, for example if a lower signal to noise ratio is required, or the polymer has a smaller modification footprint.

The modification information 20 represents a sequence of estimates of modification statuses of subject polymer units of the portion.

The portion is the portion that has partially translocated through the pore. The subject polymer units may be individual polymer units of the polymer. Alternatively, the subject polymer units may comprise polymer units forming part of a predetermined motif of polymer units. For example, where the polymer is DNA the predetermined motif of polymer units may be a cytosine nucleotide followed by a guanine nucleotide in the sequence of nucleotides along a 5′→3′ direction.

The modification statuses are statuses of modification with respect to at least one canonical type of polymer unit. The canonical type of polymer unit may be, for example, an unmodified polymer unit.

The estimates may include an estimate for each of the subject polymer units of the portion, but this is not essential. For example, the sequence of estimates may comprise estimates for each of a plurality of k-mers in the portion. The estimates of modification statuses may comprise scores in respect of the subject polymer units. The scores may represent a probability that the subject polymer unit is modified. The scores may be normalised, for example to fall between 0 and 1 or be expressed as a percentage.

Preferably, the modification statuses are methylation statuses. Methylation is a common modification that can be used to distinguish the source of a polymer, particularly biological polymers such as DNA. Methylation can also be indicative of other statuses, such as a disease condition.

By way of non-limitative example, where the polymer is DNA and the polymer units are nucleotides, then the canonical polymer unit may be cytosine or adenosine. In this example, the subject polymer units are cytosine nucleotides and the methylation statuses are statuses of methylation to at least one of 5-methyl-cytosine or 5-hydroxymethyl-cytosine, and/or the subject polymer units are adenosine nucleotides and the methylation statuses are statuses of methylation to 6-methyl-adenine.

To consider this more generally, the modified bases 5-methylcytosine (5mC) and 5-hydroxymethyl-cytosine (5hmC) are well-known epigenetic mark that regulates transcription of the genome (the switching on and off of the mechanism by which DNA is copied into messenger RNA (mRNA), which is involved in protein synthesis. Accordingly, methylation is a type of modification that the modification information 20 may represent and is important because it is generally the most biologically relevant. In cases where methylation is the desired modification, the modification information 20 may be termed methylation information.

Although methylation is a modification of particular interest, the method is not limited to determining methylation status. The modification statuses may alternatively or additionally be oxidation statuses. For example, oxidation of methylated cytosine (5-mC) 25 to 5-hydroxymethylcytosine (5-hmC), 5-formylcytosine (5-fC), 5-carboxylcytosine (5-caC), and methylation of adenine (A) to N6-methyladenine (6-mA), which are being identified as important epigenetic regulators.

In the case that the polymer is RNA, modifications are more prevalent and recent work has shown that it plays a role in regulating mRNA stability. The stability of mRNA effects control of gene expression and can effect various cellular and biological processes. To date, hundreds of RNA modifications have been characterized and may be represented by the modification information 20. Non-limitative examples include N6-methyladenosine (m6A), Inosine (I), N6,2′-O-dimethyladenosine (m6Am), 8-oxo-7,8-dihydroguanosine (8-oxoG), pseudouridine (Ψ), 5-methylcytidine (m5C), and N4-acetylcytidine (ac4C), have been shown to regulate mRNA stability and function.

The modification information 20 may represent modification statuses comprising a single chemical modification with respect to the canonical type of polymer unit, for example 5mC modification of the Cytosine DNA base as mentioned above. Alternatively, the modification information 20 may represent a combination of such modifications for a single polymer unit or multiple such polymer unit. For example, for DNA, combinations of modifications may include 5mC, 5mC+5hmC, 5mC+6 mA, 5mC+5hmC+6 mA. This further applies to the consideration of chemical modifications of the canonical polymer units present in specific context(s) (e.g., 5mC modification in the CG context), across all contexts (i.e., all positions of canonical bases across sequenced polymer), as well as any combination of them.

The method may further comprise determining sequence information 30 representing estimates of the identities of the polymer units of the portion of the sequence of polymer units. Similarly as for the estimates of the modification statuses, the estimates of the identities of the polymer units may comprise scores in respect of each of a set of types of polymer units.

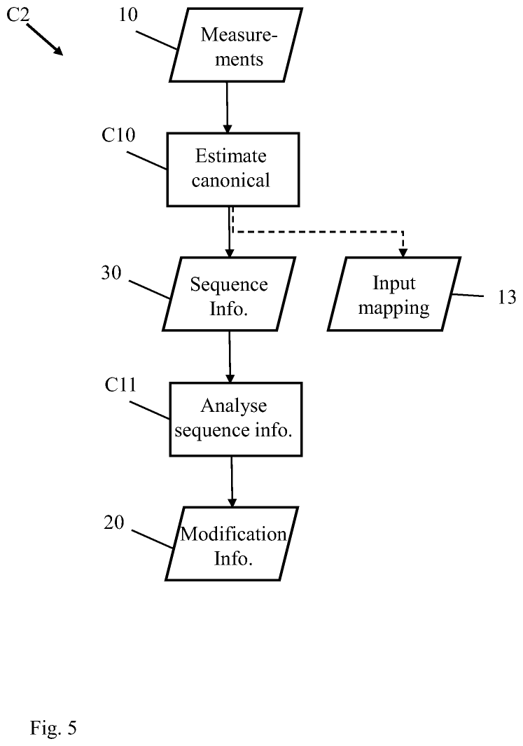

FIG. 5 illustrates an example method for determining modification information 20 that comprises determining sequence information. The method of FIG. 5 may be used in step C2 of the method of FIG. 4. In this example, the method uses an initial machine learning system in step C10 to determine sequence information 30 comprising canonical types of polymer units. The estimates of the identities of the polymer units are therefore estimates in respect of a set comprising canonical types of polymer units. As discussed above, the canonical types of polymer units may be, for example, unmodified polymer units.

A subsequent machine learning system is then used in step C11 to determine the modification information 20 from the derived sequence information 30. This means that in this example, determining the modification information 20 comprises analysing the sequence information 30 to determine the modification information 20.

In more detail, at step C10 of FIG. 5, the measurement signal 10 is supplied as an input to an initial machine learning system, which is trained to provide an output that is sequence information 30. In general, the initial machine learning system may take any suitable form, but is typically a neural network. For example, the initial machine learning system may be a neural network of the type disclosed in: Hochreiter, S. and Schmidhuber, J., 1997. Long short-term memory. Neural computation, 9(8), pp. 1735-1780; Cho, K., Van Merriënboer, B., Bahdanau, D. and Bengio, Y., 2014. On the properties of neural machine translation: Encoder-decoder approaches. arXiv preprint arXiv:1409.1259; Kriman, S., Beliaev, S., Ginsburg, B., Huang, J., Kuchaiev, O., Lavrukhin, V., Leary, R., Li, J. and Zhang, Y., 2020, May. Quartznet: Deep automatic speech recognition with id time-channel separable convolutions. In ICASSP 2020-2020 IEEE International Conference on Acoustics, Speech and Signal Processing (ICASSP) (pp. 6124-6128). IEEE; or Teng, H., Cao, M. D., Hall, M. B., Duarte, T., Wang, S. and Coin, L. J., 2018. Chiron: translating nanopore raw signal directly into nucleotide sequence using deep learning. GigaScience, 7(5), and to which standard training techniques are applied.

The sequence information 30 determined in step C10 may be a categorical output. It may represent an estimate of the identity of polymer units in the sequence between categories comprising a set of predetermined canonical polymer units. For example, in the case that the polymer unit is a DNA polynucleotide, the canonical nucleotides may be the four bases adenine (A), cytosine (C), guanine (G), and thymine (T). In general, such a categorical output may be implemented as a vector of probabilities over the categories. However, for use in the subsequent method, a hard call is made. That is the most likely category, e.g. the most likely canonical polymer unit is selected and represented in the sequence information 30.

Optionally, the initial machine learning system may also output an initial mapping (also referred to as an input mapping) 13 between the measurement signal 10 and the sequence information 30. Typically, such an initial mapping 13 is inherently generated during the operation of a machine learning system such as a neural network. It is often referred to as the “move table” in nanopore basecalling documentation and prior art. Generally, this initial mapping 13 is discarded as the generally desired output is simply the sequence estimate. However, generally the initial mapping 13 can be obtained and output from the initial machine learning system 11, when needed.

The initial mapping 13 simply describes the originating position of each polymer unit of the sequence information 30 with corresponding samples of the measurement signal 10. The initial mapping 13 may be encoded in several equivalent forms. For example, an array of indices the length of the sequence information 30 and with elements corresponding to the position of samples of the measurement signal 10 would completely represent this mapping. Equivalently the length, in number of signal positions, of each polymer unit of the sequence information 30 would completely describe this mapping in a more compact manner.

It is assumed that the position of a polymer unit within the measurement signal 10 is not before the position of the polymer unit. In other words, a polymer unit later in the sequence information 30 may not be assigned a position earlier in the measurement signal 10. It is also assumed that each input sequence polymer unit is assigned a starting position within the signal array, implying that many signal positions may be assigned to a single sequence base, and this is often the case.

As an alternative to the initial mapping 13 being output from the initial machine learning system 11, the initial mapping 13 may be derived from the measurement signal 10 and the sequence information 30 themselves. Several methods are described in the prior art for the generation of such a sequence-to-signal mapping, for example in: Stoiber, M. H. et al. De novo Identification of DNA Modifications Enabled by Genome-Guided Nanopore Signal Processing. bioRxiv (2016); or Simpson, Jared T., et al. “Detecting DNA cytosine methylation using nanopore sequencing.” nature methods 14.4 (2017): 407-410. Such methods may be applied here.

Having determined the sequence information, the method of FIG. 5 proceeds to step C11, which comprises analysing the sequence information 30 to determine the modification information 20.

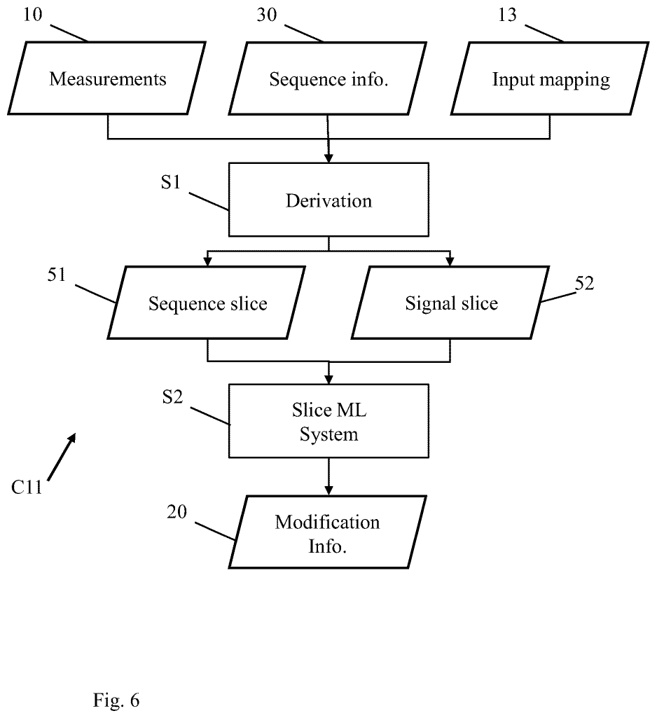

FIG. 6 illustrates a method for analysing the sequence information in step C11, which uses a slice machine learning system to derive modification information 20 from the sequence information 30. In this example, there are three inputs, namely 1) the measurement signal 10, 2) the sequence information 30, and 3) an input mapping 13 between the measurement signal 10 and the sequence information 30.

In derivation step S1, there are derived two slices, namely 1) a sequence slice 51 and a signal slice 52, which are input into the slice machine learning system. The sequence slice 51 is derived from a slice of the sequence information 30 around a subject polymer unit in the sequence of polymer units. The signal slice 52 is a slice of the measurement signal 10. Importantly, the sequence slice 51 and the signal slice 52 are mapped to each other by the input mapping 13 between the measurement signal 10 and the sequence information 30.

To summarise this at a high level, this method involves the input of a sequence slice 51, which is a canonical sequence (i.e. a sequence indicating the canonical type of each polymer unit in the portion), and a measurement slice 52 of the measurement signal 10, which is a raw measurement signal, directly into the slice machine learning system. This may be referred to as a multi-headed input. In contrast, known canonical basecalling systems are typically based on a single-headed neural network as only a single form of data is input into the neural network, namely the raw nanopore signal. To enable multi-headed input, the sequence slice 51 and the signal slice 52 are presented in a manner described further below.

The method may be applied to a single subject polymer unit in the sequence information 30 or repeatedly to plural subject polymers being all or any subset of the polymer units in the sequence information 30. For example, the method may performed for a subject polymer unit forming part of a predetermined motif comprising plural canonical polymer units. Often a motif (a short pattern of polymer units e.g. nucleotides) may include positions of ambiguity allowing several polymer units or variable widths of polymer units used to identify the relevant subject polymer units. For example, the “CG” motif, also referred to as a CpG site, is the most common motif in which methylation occurs in most mammals, and may form a motif used herein.

Examples of the derivation of the sequence slice 51 and the signal slice 52 in derivation step S1 will now be described in more detail. As mentioned above, the sequence slice 51 is derived from a slice of the sequence information 30 around a subject polymer unit and the signal slice 52 is a slice of the measurement signal 10, the sequence slice 51 and the signal slice 52 being mapped to each other by the input mapping 13. There are various ways to achieve this, for example as follows.

The measurement signal 10, the sequence estimate 30, and the input mapping 13 may be provided as a full sequencing read corresponding to the entire portion of the sequence obtained during the partial translocation. However, this may be relatively long depending on the configuration of the system, for example consisting of tens to thousands of individual polymer units for some types of sensor device 2. However, derivation step S1 provides the sequence slice 51 and the signal slice 52 with corresponding lengths that are selected to provide suitable accuracy for the slice machine learning system 41. This may be all or a part of the portion of the polymer that has (so far) translocated through the nanopore during the partial translocation.

In one approach, the signal slice 52 is a predetermined length of the measurement signal 10 around a position in the measurement signal 10 that is mapped to the subject polymer unit. In this case, once subject polymer unit within the sequence estimate 30 is identified, the location within the measurement signal 10 to which the subject polymer unit is assigned from the input mapping 13. The center of this stretch of the measurement signal 10 is defined as the center of the region of interest. From this position a fixed width of signal is extracted using a user defined range before and after this position.

In this case, the predetermined length of the measurement signal 10 may, for example, be in a range from 20 sample points to 1000 sample points, for example 100 sample points. Larger lengths of the measurement signal 10 may be more than 1000 sample points. The signal slice 52 may be arranged symmetrically or asymmetrically around the sample point that is mapped to the subject polymer unit.

In addition to extracting the signal slice 52 from this region, the sequence slice 51 is selected as the polymer units mapped to the stretch of the signal slice 52 by the input mapping 13. Accordingly, the length of the sequence slice 51 varies for different subject polymer units.

In another approach, the sequence slice 51 is a predetermined length of the sequence estimate 30, i.e. a predetermined number of polymer units. In this case, once the sequence slice 51 has been extracted, the signal slice 52 is derived as the portion of the measurement signal 10 that is mapped to the sequence slice 51 by the input mapping 13. Accordingly, the length of the signal slice 52 varies for different subject polymer units.

In this case, the predetermined number of polymer units may be in a range from 1 polymer unit to 100 polymer units. The range of polymer units to be considered may be dependent on the type of nanopore used.

Optionally, the sequence slice 51 may be selected to consider nanopore kinetics, as follows. When the rate of translocation of a polynucleotide through a nanopore is controlled by a molecular brake in the form of an enzyme, it is believed for example that modified bases affect the enzyme kinetics such as the kinetics of unwinding of double stranded polynucleotides by certain helicases. In the case of a helicase as the binding enzyme which may serve to unwind double stranded DNA and control passage of a resultant single stranded DNA strand through the nanopore, consideration of those nucleotides within the enzyme binding region may further provide information about the signal.

As such, it may be of value to provide such information to nanopore modified base detection algorithms. This may be achieved by the sequence slice 51 being derived in a manner that one or more nucleotides of the sequence slice 51 are within a region of the enzyme acting as a molecular brake to controls translocation of the polymer.

This may improve accuracy compared to providing the same size of signal, but without including the signal when the base of interest is in the molecular brake. Note that this may provide improved performance over alternative nanopore modified base detection algorithms which attempt to provide this information via summaries of the raw nanopore signal as signal to sequence assignment/alignment algorithms are often quite error prone. As noted in other sections passing the raw nanopore signal into the neural network may allow for improved performance bypassing issues with sequence to signal alignments.

It has been shown that changes in the signal may be influenced most due to interaction of the nucleotides with one or more constrictions of the nanopore, a constriction being a region of the internal lumen of the nanopore of narrow cross-section, see for example, FIG. 1 of Butler et al, Proceedings of the National Academy of Sciences 105 (52), 20647-20652 which shows an MspA nanopore with an inner narrow constriction at the D90N/D91N region and FIGS. 1 and 2 of WO2016/034591 which shows the inner constriction region of CsgG nanopore. However interaction with other regions of the nanopore can affect the signal and nucleotides external to the nanopore are also believed to have an influence on the measured signal. In use, the binding enzyme is typically held against the cis or trans opening of the nanopore during translocation of the polynucleotide through the nanopore under an applied potential. Thus nucleotides immediately outside of the lumen of the nanopore are typically within the region of the binding enzyme, for example with dDA helicase as the polynucleotide binding enzyme and CsgG as the nanopore, the distance between the enzyme and the constriction is estimated at between 10 and 14 bases (or approximately 100 to 140 signal points). Signal point measurements depend on several factors and may vary drastically from these values for other pore chemistries).

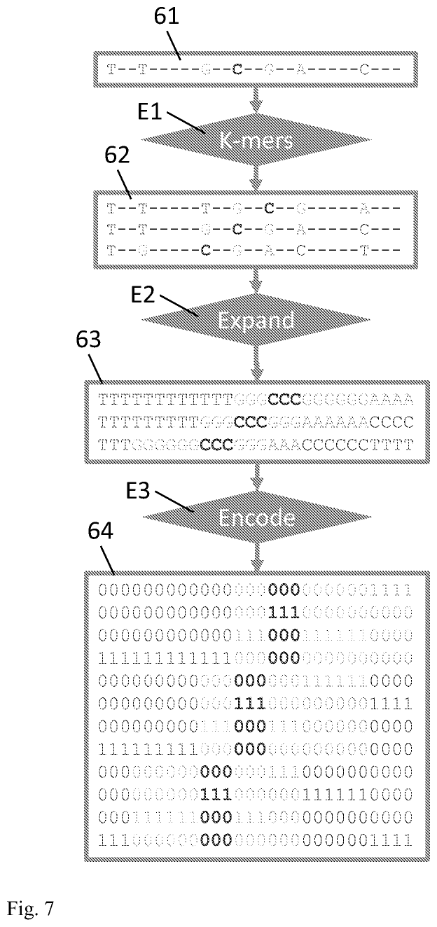

FIG. 7 illustrates a particular method of generating the sequence slice 51 in an appropriate form for input to the slice machine learning system mapped to the signal slice 52. This procedure is intended to maximize the information presented to the slice machine learning system.

Initially, a first signal slice 61 is extracted as a slice of the sequence estimate 30, which, for non-limitative, illustrative purposes, has in FIG. 7 a particular sequence of nucleotides that are different canonical nucleotides selected from the four bases A, C, G or T. Graphically in FIG. 7 the input mapping 13 is represented by dashes. In particular, each element of the first sequence slice 61 that is either a nucleotide or a dash corresponds to a respective sample point in the corresponding signal slice 52, in accordance with the input mapping 13.

In step E1, the first sequence slice 61 is encoded into a second sequence slice 62 by replacing each polymer unit by a respective k-mer, so that the second sequence slice 62 is a sequence of k-mers corresponding to respective polymer units in the first input slice 61. Thus, compared to the first sequence slice 61, the second sequence slice 62 has the same length but increased dimensionality so that each element of the second sequence slice 62 is a vector of k dimensions (k being 3 in FIG. 7, by way of non-limitative example). Each k-mer in the second sequence slice 62 comprises a group of k polymer units (arranged vertically in FIG. 7), where k is a plural integer. Each k-mer includes a) the respective polymer unit (along the middle dimension in FIG. 7), and b) (k−1) polymer units that are adjacent to the respective polymer unit in the sequence estimate 30. The (k−1) adjacent polymer units symmetrical around the respective polymer unit in FIG. 7, but as an alternative (k−1) adjacent polymer units be selected asymmetrically. It should be noted that this encoding requires a fixed number of polymer units before and after the first signal slice 61 to enable the construction of the k-mers.

This change from polymer units to k-mers effectively provides additional contextual information to the individual polymer. These k-mers may be thought of as representing the portion of the polymer which physically interacted with the nanopore at a particular position within the signal during the partial translocation, although that is conceptual and may not be a complete description of any particular sensor device 2. Nonetheless, in the case that the polymer translocates through the nanopore, k may have has a value selected so that the length of the k-mer is greater than the length of the nanopore lumen through which the polymer translocates.

The use of k-mers in this way has been shown to improve the accuracy of the estimation performed by the slice machine learning system. In general, the k may have any value that provides such an improvement, noting that increasing k increases the size of the data without significantly increasing the computational cost. In some examples, k may have a value in a range from 3 to 50, but higher values are also possible.

As an alternative, step E1 may be omitted so that the following steps are performed on the first sequence slice 61, although that is likely to reduce the accuracy of the estimation performed by the slice machine learning system.

In step E2, the second sequence slice 62 is expanded into a third sequence slice 63, so that it has the same length as the signal slice 52. In this example, the expansion is performed by repetition padding which is shown graphically in FIG. 7 as a replacement of the dashes by the k-mer that preceded them. This expansion allows efficient design of the slice machine learning system, described below.

In step E3, the third sequence slice 63 is binary encoded into a final sequence slice 64, which is used as the input sequence slice 51 to the slice machine learning system. The binary encoding encodes each polymer unit in binary format, in this example using a one-hot encoding (“1000” for A; “0100” for C; “0010” for G; “0001” for T; and “0000” for unknown or missing bases). For each position in the third sequence slice 63, the k vectors of length 4 for the k polymer units of the k-mer are concatenated to form a vector of length 4 k.

The slice machine learning system is supplied with the sequence slice 51 and the signal slice 52 of equal length as a double-headed input. The slice machine learning system has been trained to provide an output 80 representing an estimate of the modification status of the subject polymer unit with respect to at least one canonical type of polymer unit. The output 80 is a categorical output. That is, the output 80 estimates the identity of the subject polymer unit as between a set of categories, namely between modified and non-modified forms of the at least one canonical type of polymer unit. Such a categorical output 80 may be implemented as a vector of probabilities over the categories. The slice machine learning system is trained to maximise the probability for the correct output category and minimise the probability for the incorrect output categories. To optimize categorical output type, the cross-entropy loss is generally used in the slice machine learning system that is described further below, although there are other loss functions that could be applied to such a categorical output 80. A sequence of outputs 80 for a plurality of subject polymer units may be used directly as the modification information 20. Alternatively the sequence of outputs 80 may be further processed to derive the modification information 20. For example the modification information 20 may represent only subject polymer units in the polymer that are modified.

The categories represented by the output 80 are a canonical polymer unit and at least one modified form of the canonical polymer unit.

In general, the slice machine learning system may use a variety of different machine learning techniques. However, a particularly advantageous form the slice machine learning system is as a neural network.

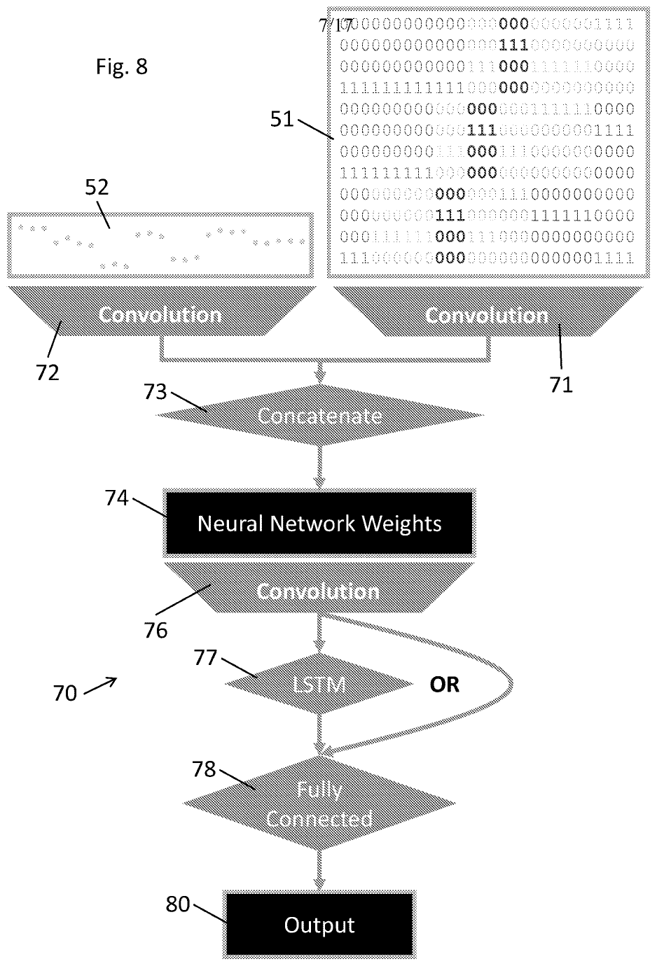

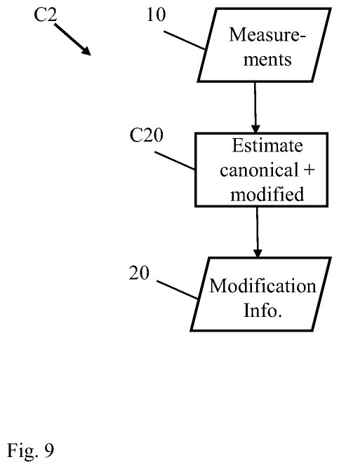

By way of illustration, FIG. 8 shows an example in which the slice machine learning system is a neural network 70. There will now be described the features or components of the neural network 70 and training methods for such a neural network.

The neural network 70 comprises a first input stage 71 to which the sequence slice 51 is supplied, and a second input stage 72 to which the signal slice 52 is input.

The first input stage 71 comprises at least one first input neural network layer. The input neural network layer(s) of the first input stage 71 may be convolutional neural network layer(s).

The second input stage 72 also comprises at least one second input neural network layer. The input neural network layer(s) of the second input stage 72 may be convolutional neural network layer(s).

The outputs of the first and second input stages 71 and 72 are supplied to a concatenation layer 73 which concatenates those outputs to provide a concatenated output that is supplied to the remaining layers, also comprising at least one convolutional neural network layer. The concatenation is performed feature-wise so that the temporal (sequencing signal time direction) correspondence between inputs to the concatenation layer 73 derived from the sequence slice 51 and the signal slice 52 is preserved. Output values from the concatenation layer 53 are then further processed by layers in the neural network 50 as a single input.

The further layers are arranged as follows.

The concatenated output 74 is supplied to a combined convolutional neural network stage 76 that comprises at least one convolutional neural network layer.

The convolutional neural network layers of the first and second input stages 71 and 72 and the combined convolutional neural network stage 76 may be of conventional construction. Such convolutional neural network layers are well known in the art, but in summary operates on fixed sized moving windows of the input data at a stride along the input data. At each window, the input features are matrix multiplied by a set of weights to produce the outputs of the layers.

Each of the first and second input stages 71 and 72 and the combined convolutional neural network stage 76 may include any number of convolutional layers stacked together, with different hyper-parameters being applied at each layer including window size, stride, and number of parameters/weights. Convolutional layers may each be followed by a batch normalization layer and an activation function (in this case swish nonlinearity) as well as other standard neural network components. The convolutional layers in the first and second input stages 71 and 72 are designed to produce the same output size in terms of the length and feature dimensions. Note that the input for each of the the first and second input stages 71 and 72 has a different feature dimension size.

No padding is used with any of the convolutional layers as is common in some fields of machine learning when using convolutional layers.

The output of the combined convolutional neural network stage 76 is supplied to a LSTM (long short-term memory) stage 77 comprising at least one LSTM layer, which is an example of a recurrent neural network (RNN) layer, and may be of conventional construction.

The LSTM stage 77 is optional and may be omitted.

The output of the LSTM stage 77, or the output of the combined convolutional neural network stage 76 in the event that the LSTM stage is omitted, is supplied to a fully connected stage comprising at least one fully connected layer, which again may be of conventional construction.

A description of recurrent neural network layers that may be applied in the LSTM stage 77 and the fully connected stage is given in Sak, H., Senior, A. W. and Beaufays, F., 2014. Long short-term memory recurrent neural network architectures for large scale acoustic modeling.

The neural network 70 processes the input in batches. The cross-entropy loss is calculated for each batch, as described above. An optimizer is used during training to backpropagate. In one demonstration the optimizer may be the AdamW optimizer. Backpropagation is done in the standard fashion as described in prior art (Loshchilov, I. and Hutter, F., 2017. Decoupled weight decay regularization. arXiv preprint arXiv:1711.05101).

The output 80 is used as the estimate of the modification status of the subject polymer unit. The sequence of such outputs 80 for a plurality of polymer units may be used as the modification information 20 in the method of FIG. 4. Alternatively, the sequence of outputs 80 may be processed to generate the modification information 20, for example to generate a dense representation of the modified units of the polymer.

FIG. 9 illustrates an alternative method of analysing the measurements in step C2 of FIG. 4 to determine the modification information 20 in respect of the portion of the sequence of the polymer units.

In the method of FIG. 9, the estimates of the identities of the polymer units represented by the sequence information 30 are estimates in respect of a set comprising canonical types of polymer units and one or more modified forms of at least one canonical type of the polymer units. The modification information 20 then comprises the estimates in respect of the modified forms of the at least one canonical type of the polymer units. For example, where the polymer is DNA, the set in the method of FIG. 5 may comprise the canonical nucleotide bases adenine (A), cytosine (C), guanine (G), and thymine (T) as well as the modified bases 5-methylcytosine (5mC) and 5-hydroxymethyl-cytosine (5hmC). The sequence information 30 would then comprise estimates in respect of each of these canonical and modified bases. The estimates in respect of the modified bases 5-methylcytosine (5mC) and 5-hydroxymethyl-cytosine (5hmC) would be taken as the modification information 20.

In this method, the identities of the polymer units are directly estimated as being of a canonical (i.e. unmodified) type or a modified form of the canonical type. This simplifies the estimation because only a single stage of processing is required to obtain the modification information 20, in comparison to the two-stage method of FIG. 5. However, in some situations, this method may have reduced accuracy compared to the two-stage method due to the greater difficulty in reliably distinguishing between a larger number of different categories in a single step. As for the method of FIG. 5, a machine learning algorithm such as a neural network may be used to estimate the identities of the polymer units.

Returning to the method of FIG. 4, following the analysis in step C2 of the measurements to determine the modification information 20, the method comprises a step C3 of classifying the polymer.

In step C3, the modification information 20 collected in step C2 is used to classify the polymer as belonging to one of a set of classes based on the modification information 20. The classification may be performed by any suitable method. For example, the modification information 20 may be passed to a machine learning classifier, as described in more detail below. However, the method (and in particular the step of classifying the polymer) does not comprise comparing the modification information 20 to a reference polymer sequence.

It may be possible to classify the polymer by comparing the modification information 20 to a database of known reference sequences and, for example, determining a similarity to exemplary reference sequences for particular classes. However, this requires a large database of reference polymer sequences, which can consume a lot of memory and processor time for the comparison. In addition, using reference polymer sequences reduces the ability of the method to classify uncommon polymers that do not match well to any reference polymer sequences. Therefore, in the present invention, the classification is performed based on the modification information 20 without use of a reference polymer sequence (for example based only on the modification information 20).

Where the polymer derives from an organism, the classes of the set of classes may be taxonomic domains or kingdoms. For example, a first class of the set of classes may be bacterial organisms or a type of bacterial organism, and a second class of the set of classes may be eukaryotic organisms or a type of eukaryotic organism.

At least one class of the set of classes may represent a target sequence or class of target sequences. For example, the target sequence may be a polymer from a eukaryotic organism such as a mammal or human. At least one class of the set of classes may represent a background sequence or class of background sequences. For example, the background sequence may be a polymer from a bacterial organism.

While these are common examples, the classes of the set of classes are not limited to being based on an organism from which the polymer is derived. For some applications, the classes may represent a type or condition of the polymer, for example a modification profile of the polymer. In this case, the polymers of different classes may be derived from organisms of the same taxonomic domain or kingdom, or even from the same organism. For example, as will be discussed further below, another application for which the present method is advantageous is detection of artificially-methylated regions for protein binding site identification and affinity analysis. For this application, the classes may represent whether or not the polymers have a particular modification profile, in this example the artificially-induced methylation profile.

The target sequence and background sequence are not specific reference sequences (e.g. from a database) against which the modification information 20 is compared. They are rather types of sequence that are of interest for sequencing by the biochemical analysis system 1 (target sequence) or are not of interest (background sequence).

The method of FIG. 9 may use the techniques disclosed in WO-2020/109773, which is incorporated herein by reference.

As mentioned above, classifying the polymer may be carried out by any suitable method. Classifying the polymer as belonging to one of the set of classes may comprise inputting the modification information 20 into a machine learning classifier that classifies the polymer as belonging to one of the set of classes based on the modification information 20. The machine learning classifier may comprise a neural network.

Preferably, the machine learning classifier is trained using modification information 20 in respect of plural classes. This will increase the accuracy and precision of classification by providing additional information to differentiate the plural classes. However, this is not essential, and in some cases the machine learning classifier may be trained using modification information 20 in respect of only a subset of fewer than all of the classes in the set of classes. The machine learning classifier may be trained using modification information 20 in respect of only a single class of the set of classes, for example the class representing a target sequence or the class representing a background sequence.

The method uses a pre-trained machine learning classifier to classify the polymer. The machine learning classifier is trained on data external to the data being sequenced. For example, the modification information 20 used for training may be obtained from analysis of samples of known composition, or may be derived from external databases of previously-sequences polymers.

In some embodiments, the polymer may be classified as belonging to one of the set of classes based on the modification information 20 only. A specific example of such an embodiment where the modification information 20 comprises 5mC methylation probabilities for CG motifs in DNA is as follows.

The 5mC CG-context-localized methylation probabilities are aggregated across all CG motifs within the portion of the sequence of the polymer obtained during the partial translocation. In this example, the sequential order of the methylation probabilities is not significant, and so the classification is not dependent on an order of the modification statuses of the subject polymer units. However, in other examples the classification may depend on the order of the modification statuses of the subject polymer units.

The integer range of 1-255 is split into N equal-sized intervals (e.g., N=10 results in intervals [1-25][26-51] . . . [231-255] and the frequency of methylation probabilities values within a specific interval is counted. For example, a methylation probability of 13 triggers a counter increase for the interval 1-25 as 1<=13<=25. The counted values are then normalized (i.e., divided) by the total number of the CG motifs in the observed sequence. The resulting N-sized vector of real number values between 0 and 1 (the sum of which N values totals 1.0) represents the 5mC CG methylation probability tabular frequency profile, which may be referred to as the methylation profile. Besides the methylation profile, the portion of the sequence is further characterized by the overall length of the portion in polymer units (integer number) and the number of CG motifs (integer number). These 2+N integer and real numbers are referred to as the sequence tabular methylation profile. Consequently, the classification of the polymer depends on one or more (preferably all) of the length of the portion, the number of subject polymer units (in this example, the number of CG motifs), and the estimate of the modification status for each of the subject polymer units.

While the number of CG motifs and the length of the portion of is utilized in the sequence tabular methylation profile representation, this does not necessary require the canonical sequence information to be used. The method can be designed to determine the estimates of modification status (e.g. 5mC methylation probability) for every subject polymer unit (e.g. CG motif) of the portion of the sequence from the measurement signal alone (as discussed above). The resulting modification information 20 (e.g. the vector of methylation probabilities) then provides both the number of subject polymer units and the estimates of modification without requiring canonical basecalling of the portion of the sequence of polymer units. Furthermore, the sequence length of the portion can also be estimated from the measurement signal without performing canonical basecalling.

Following the above pre-processing to obtain the sequence tabular methylation profile, the sequence tabular methylation profile is supplied to a pre-trained machine learning classifier (e.g. regularizing gradient boosting framework XGBoost). The classifier outputs a probability (for example in the form of a real number value between 0.0 and 1.0) for the polymer to be either bacterial or non-bacterial. The 0.5 probability threshold is used to translate the real number value output into the binary classification result assigning either bacterial (<0.5) or non-bacterial status (>=0.5), respectively to the polymer.

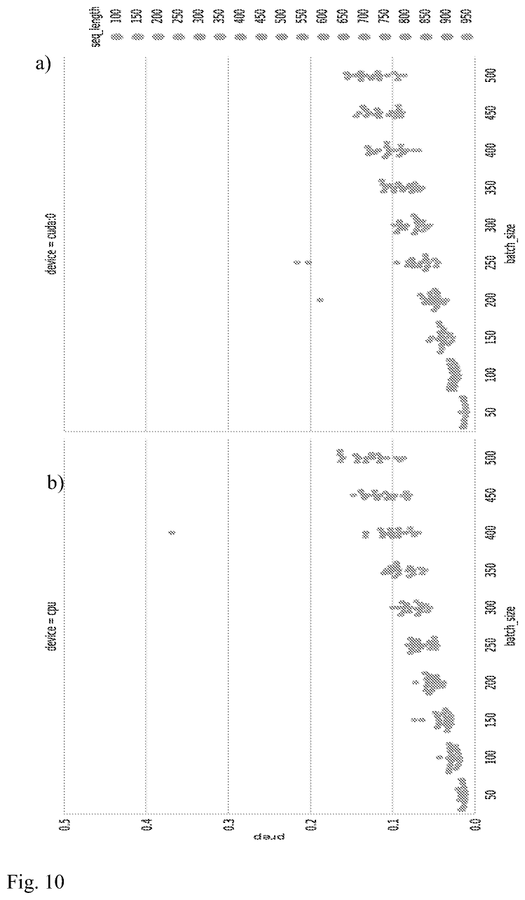

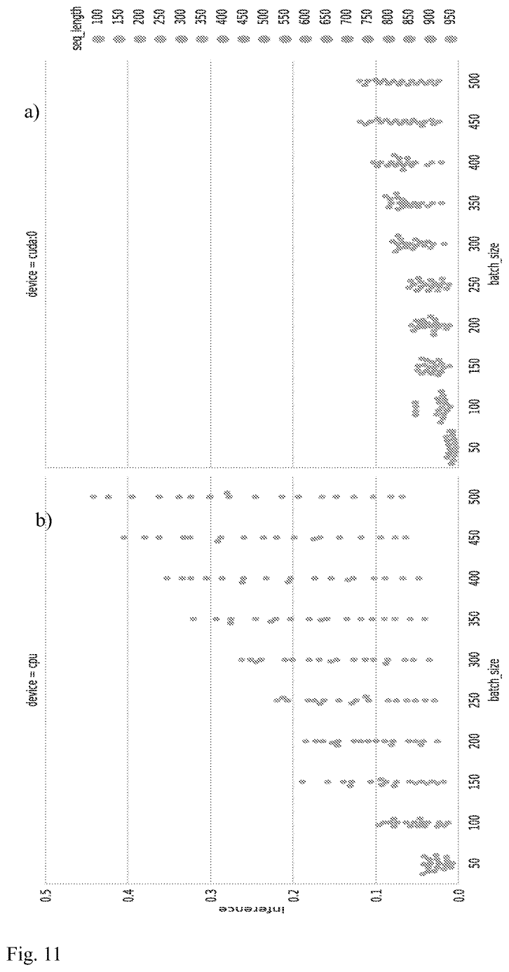

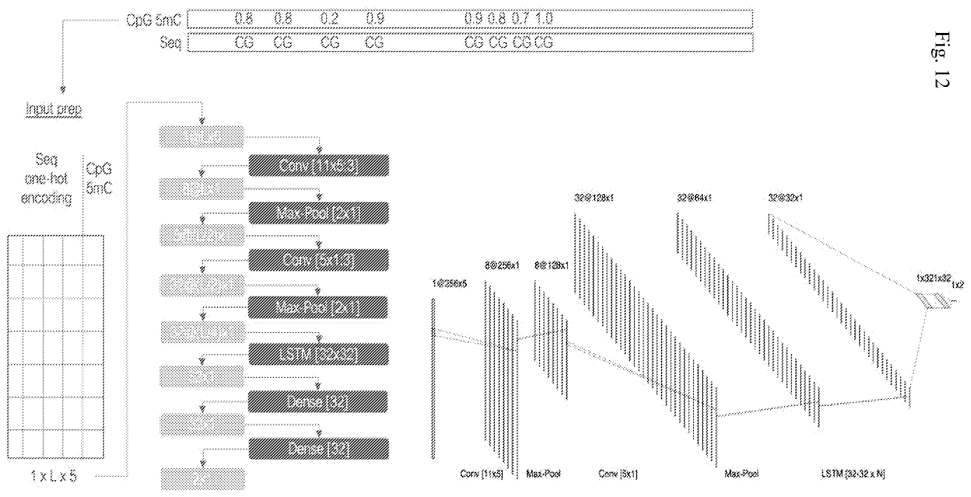

The computation performance (in seconds) of the pre-processing steps to obtain the sequence tabular methylation profile and the classification inference by the machine-learning classifier are shown in FIG. 10 and FIG. 11. FIG. 10 shows a comparison of computational performance of input data preprocessing steps on different read lengths and batch sizes when performed on a) GPU and b) CPU. FIG. 11 shows a comparison of computational performance of input data classification on different read lengths and batch sizes when performed on a) GPU and b) CPU.

As an alternative to the embodiment above, in some embodiments the polymer is classified as belonging to one of the set of classes based on the modification information 20 and the sequence information 30. A non-limiting example of such an embodiment (analogous to the above example where the modification information 20 comprises 5mC methylation probabilities for CG motifs in DNA) is as follows.

The sequence information comprising the basecalled canonical DNA sequence for the portion of the sequence of length L is encoded with a one-hot encoding into an L×4 matrix where every one of the L rows, indexed with i, is a 011 bit vector with a single one of the 4 columns, indexed with j, having a value 1 and the rest of the columns having a value 0. The location of the value 1 indicates the canonical base, with j=1 (for A), j=2 (for C), j=3 (for G), and j=4 (for T).

The 5mC CG-context-localized methylation probabilities are then represented as a L×1 floating point methylation modification vector. For each canonical base in the input sequence of length L, the value is set to 0.0 except for cytosine base in the CG context, in which case the real number/floating point value is set to the probability of the respective cytosine base being methylated (as per the inference method's output).

The methylation modification vector and the sequence one-hot encoding matrix are then concatenated to form a L×5 matrix with the first 4 columns representing the one-hot encoding of the input basecalled canonical DNA sequence (as described above), while the 5th column represents the 5mC Cg methylation profiles of the portion of the sequence. The total L×5 matrix is referred to as the input representation.

FIG. 12 demonstrates how the input representation is processed via an exemplary trained neural network. The neural network of FIG. 12 is comprised of 2 convolution+max-pool layers, followed by a recurrent neural network layer with long short term memory (LSTM) modules, followed by two fully-connected layers, ending with a softmax layer with 2 output neurons suitable for binary classification.

The preprocessing to obtain the input representation and the neural network architecture encoding can be implemented with any suitable method or framework, for example a PyTorch programming framework. Similarly, the neural network training can be performed with any suitable method of framework, for example a skorch programming framework.

Returning again to FIG. 4, after classifying the polymer in step C3, the method comprises operating the biochemical analysis system 1 to reject the polymer or continue taking measurements from the polymer based on the class to which the polymer unit is classified as belonging. This is carried out by steps C4, C5, and C6.

In step C4, a decision is made responsive to the classification of the polymer in step C3 either (a) to reject the polymer being measured (i.e. eject the partially-translocated polymer from the nanopore), or (b) to continue taking measurements (i.e. sequencing the polymer) until the end of the polymer. Having made the decision in step C4, a feedback signal is sent to the biochemical analysis system 1 to control the operation to continue taking measurements or reject the polymer. The feedback signal may be referred to as an enrichment feedback signal (if the decision is to continue taking measurements) or a depletion feedback signal (if the decision is to reject the polymer).

In some examples, the decision may alternatively be taken that further measurements are needed to make a decision. This option may be taken, for example, if a certainty in the classification of the polymer is below a predetermined threshold. In this case, the method will not proceed to step C5 or C6, but will instead return to step C2 once further measurements have been taken. The analysis of the measurements and classification of the polymer will then be repeated and the decision step C4 revisited.

Where at least one class of the set of classes represents a target sequence or class of target sequences, operating the biochemical analysis system 1 to reject the polymer or continue taking measurements from the polymer may comprise operating the biochemical analysis system 1 to continue taking measurements when the class to which the polymer unit is classified as belonging by the machine learning classifier is said at least one class representing a target sequence. This means that sequencing continues if the polymer is identified as belonging to a class for which reads which should be enriched.

Where at least one class of the set of classes represents a background sequence or class of background sequences, operating the biochemical analysis system 1 to reject the polymer or continue taking measurements from the polymer may comprise operating the biochemical analysis system 1 to reject the polymer when the class to which the polymer unit is classified as belonging by the machine learning classifier is said at least one class representing a background sequence. This means that sequencing is stopped if the polymer is identified as belonging to a class for which reads which should be depleted.

Other criteria may also be used to determine whether the polymer should be rejected, or if measurements should continue to be taken. For example, where at least one class of the set of classes represents a target sequence or class of target sequences, operating the biochemical analysis system 1 to reject the polymer or continue taking measurements from the polymer may comprise operating the biochemical analysis system 1 to reject the polymer when the class to which the polymer unit is classified as belonging by the machine learning classifier is not said at least one class representing a target sequence. In other words, the polymer is rejected if a positive identification of the polymer as belonging to a class of target sequences is not possible. This could include if no positive identification of the polymer as belonging to any class is possible, for example if an uncertainty in the classification is above a predetermined threshold.

Analogously, where at least one class of the set of classes represents a background sequence or class of background sequences, operating the biochemical analysis system 1 to reject the polymer or continue taking measurements from the polymer may comprise operating the biochemical analysis system 1 to continue taking measurements from the polymer when the class to which the polymer unit is classified as belonging by the machine learning classifier is not said at least one class representing a background sequence.

If the decision made in step C4 is (a) to reject the polymer being measured, then the method proceeds to step C5 wherein the biochemical analysis system 1 is operated to reject the polymer, so that measurements can be taken from a further polymer.

The at least one sensor element 230 may be operable to eject a polymer that is translocating through the nanopore 232. In this case, operating the biochemical analysis system 1 to reject the polymer comprises operating the sensor element 230 to eject the polymer from the nanopore 232 and accept a further polymer in the nanopore 232.

Any suitable method may be used to eject the polymer. For example, the at least one sensor element 230 may be operable to eject a polymer that is translocating through the nanopore 232 by application of an ejection bias voltage sufficient to eject the polymer. In this case, operating the sensor element 230 to eject the polymer from the nanopore 232 is performed by applying an ejection bias voltage.

For example, considering the biochemical analysis system 1 described above, the electronic circuit 4 may apply a bias voltage across the nanopore 232 of the sensor element 230 that is sufficient to eject the polymer 233 currently being translocated. This ejects the polymer 233 and thereby makes the pore 232 available to receive a further polymer. After such ejection in step C5, the method may return to step C1 and so the electronic circuit may apply a bias voltage across the pore 232 of the sensor element 230 that is sufficient to enable translocation of a further polymer through the pore 232. This method is particularly convenient, because it can make use of the same electronic circuit 4 that is used to apply the bias voltage to promote translocation of the polymer for measurement. This removes the need to supply additional hardware functionality to enable the use of the present method, thereby allowing the present method to be deployed in existing devices.

In some alternative examples, in step C5 the biochemical analysis system 1 is caused to cease taking measurements from the currently selected sensor element 230 and instead take measurements from a different sensor element 230. At the same time, in step C5, the electronic circuit 4 is controlled to apply a bias voltage across the pore 232 of the sensor element 230 that is sufficient to eject the polymer 233 currently being translocated through the currently selected sensor element 230 so that sensor element 230 is available to receive a further polymer in the future. The method then returns to step C1 which is applied to the newly selected sensor element 230 so that the biochemical analysis system 1 starts taking measurements therefrom.

If the decision made in step C4 is (b) to continue taking measurements until the end of the polymer, then the method proceeds to step C6 without repeating the steps C2 and C3 so that no further chunks of data are analysed. In step C6, the sensor element 1 continues to be operated so that measurements continue to be taken until the end of the polymer. Thereafter the method reverts to step C1, so that a further polymer may be analysed.

If the decision made in step C4 is that further measurements are needed to make a decision, then the method reverts to step C2. Thus, measurements of the translocating polymer continue to be taken until a chunk of measurements is next collected in step C2 and analysed in step C3. The chunk of measurements collected when step C2 is performed again may be solely the new measurements to be analysed in isolation, or may be the new measurements combined with previous chunks of measurements. Using a larger chunk size may reduce the advantage in terms of enrichment/depletion of target/background sequences, but can increase the accuracy with which the polymers to be enriched/depleted can be identified. However, the maximum chunk size is ultimately limited by the largest size at which the underlying hardware/firmware of the biochemical analysis system can effectively ejection the partially translocated polymer from the nanopore 232.

The present method is advantageous for many applications, but in particular in the area of environmental DNA sample sequencing. Most environmental DNA samples contain a collection of cell and cell-free DNA originating from a mixture of sources. For example, fecal samples often contain host DNA mixed with bacterial DNA, the latter coming from both the host's microbiome and the surrounding environment. However, experiments conducted on the sample will often only be targeting the analysis of full-length host DNA, rather than the admixed bacterial DNA.

Host DNA analysis would benefit from increased total host DNA sequencing yield. Therefore, improving the sequencing yield of the host DNA and decreasing the sequencing yield of the non-host DNA provides a material benefit. There exist several wet-lab/library-prep protocols (e.g., https://www.nature.com/articles/s41598-018-20427-9) for enriching host DNA molecules and/or depleting bacterial DNA molecules in the library to be sequenced, which results in the standard whole-genome sequencing (WGS) experiment having a better host DNA yield.

The present method can achieve a similar improvement by increasing the ratio of host DNA sequencing yield to non-host DNA sequencing yield. This is achieved classifying the polymer as belonging to one of a set of classes according to the present method, where the set of classes includes a first class of bacterial organisms and a second class of eukaryotic organisms. This can be achieved using the modification information 20, which may comprise estimates of modification statuses such as methylation statuses.

For example, there are well characterized differences in profiles of methylation to 5-methyl-cytosine (referred to as 5mC methylation) for bacterial vs eukaryotic genomes. Eukaryotic organisms' genomes contain strong 5mC methylation modification of Cytosine base in the CG motifs, while bacterial genomes lack 5mC methylation in CG motifs. Bacterial genomes also contain methylation in other certain sequence motifs (e.g., GGWCC, where W=(G|C)) which are far more infrequent in their appearance.

The ability to classify the partially translocated DNA molecule in real time as bacterial or non-bacterial permits the present method to increase the host DNA yield during sequencing as well as decrease the non-host DNA sequencing output. As discussed above, various types of modification can be used to perform this classification. A common example is 5mC methylation, which can be both used on its own as well as jointly with other modifications such as methylation to 6-methyl-adenine (6 mA methylation). 6ma methylation is abundant in bacterial genomes while non-existent in mammalian genomes. Importantly, the present method does not require prior knowledge about either the host or non-host DNA compositions of the considered sample, as would be required when using reference sequences to classify the polymer. Rather it relies on general biological facts about modification information such as 5mC and 6 mA methylation patterns between the DNA of particular targets.

The present method differs from the previously mentioned wet-lab protocols such as methylation-based enrichment of host DNA for WGS sequencing. Specifically, the present method (i) is completely computational so that no wet-lab sample manipulation is required prior to processing by the biochemical analysis system 1, and (ii) allows for analysis of the background sequences, unlike existing wet-lab enrichment strategies. The latter difference is because even if a polymer classified as a background sequence is rejected to stop sequencing of the undesired polymer molecule, the modification information (and sequence information if determined) relating to the portion of the polymer measured during the partial translocation before rejection can still be retained. This information can be further analysed later, for example to be used for non-host DNA species-of-origin taxonomic classification and/or taxonomic abundance estimation in the original sample. The present method is therefore doubly advantageous in providing enrichment of the polymer classes of interest, while still providing a larger amount of information about the background polymer classes than existing methods.

The present method is also advantageous compared to methods that classify by comparing the portion of the sequence of the polymer to a reference polymer sequence. This is because methods using comparison to reference sequences require prior knowledge about the target sequence that is to be enriched to allow alignment-based sequence comparison to the reference sequence. In addition, if there is a large number of classes representing target sequences (e.g., analysis of DNA in a water sample aiming to enrich the DNA of all local fish species), then the number of reference sequences required is very large. This may mean that the number of reference sequences is too large for computationally effective comparison, or the reference sequences for some or all target sequences may be of poor quality or even non-existent.

Another application for which the present method is advantageous is artificially methylated regions for protein binding site identification and affinity analysis. Academic researchers have previously described several protocols for mapping protein-DNA interactions genome wide with single-molecule long read sequencing (e.g., DiMeLo-seq; https://www.nature.com/articles/s41592-022-01475-6). In the aforementioned protocol, a 6 mA methylation modification is artificially induced in the regions of a protein of interest where said protein interacts with the DNA molecules. DNA molecules are then sequenced in a WGS fashion with, for example, a nanopore sequencing platform with no adaptive sampling setup. 6 mA methylation is subsequently inferred on fully sequenced and basecalled DNA molecules. Then reference-based alignment of the sequenced molecules is performed and 6 mA methylation frequencies are considered for every region of the reference genome sequence. Due to the fact that, for example, human DNA does not have native 6 mA methylation modification, regions with observed 6 mA methylation are considered protein-interacting.

The present method can improve the analysis power and accuracy of methods such as the WGS sequencing setup proposed in DiMeLo-seq because the protein-interacting regions often do not comprise the whole reference genome sequence. The present method can enrich DNA molecules with 6 mA modification observed within the first portion of the sequence, based on the measurement taken during the partial translocation. This provides a greater yield for the protein-interacting regions, while reducing the yield for off-target regions. The present method would still allow for both qualitative and quantitative types of analyses described in the DiMeLo-seq (and similar) protocols, while enhancing their power.

Below are described results of training and validation of embodiments of the present method. In particular, results are described for 1) an embodiment in which polymer is classified as belonging to one of the set of classes based on the modification information 20 only (referred to below as pure adaptive methylation sampling, or pAMS), and 2) and embodiments in which the polymer is classified as belonging to one of the set of classes based on the modification information 20 and the sequence information 30 (referred to below as augmented adaptive methylation sampling, or aAMS). The results are based on training and validation of machine-learning classifiers using both proprietary and publicly available nanopore-sequenced DNA datasets.

The following data sets were used as shown in Table 1. Internal datasets are proprietary data sets, while external data sets are publically available data sets. The internal datasets were generated using the commercially-available Ligation Sequencing Kit SQK-LSK110 for DNA library preparation, with sequencing carried out on Oxford Nanopore Technologies equipment, specifically R9.4.1 flowcells using either a MinION or PromethION device.

TABLE 1

| Target | | In/ | |
|---|---|---|---|
| class | Organism | external | Data source |
| vertebrate | human | internal | colo829bl |
| vertebrate | frog | internal | frog |
| vertebrate | Atlantic cod | external | [https://doi.org/10.1534/g3.120.401423] |
| vertebrate | mouse | internal | mouse |
| plant | arabidopsis | external | 41rabidopsis |
| | | | [https://doi.org/10.1038/s41467-021-26278-9] |
| plant | O. sativa | external | O. Sativa |
| | | | [https://doi.org/10.1038/s41467-021-26278-9] |
| plant | maize | internal | maize |
| plant | B. nigra | external | B. Nigra |
| | | | [https://doi.org/10.1038/s41477-020-0735-y] |
| bacterial | mix | external | Dog fecal (presented at NCM 2021) |
| | | | [http://doi.org/10.1186/s12864-021-07607-0] |
| bacterial | mix | external | Cattle rumen |
| | | | [https://doi.org/10.1038/s41587-019-0202-3] |
| bacterial | equal zymo | external | equal zymo mix |
| | mix | | [https://doi.org/10.1093/gigascience/giz043] |
| bacterial | equal zymo mix + | internal | Basecalling training data |
| | c. elegans + | | |
| | human | | |
| bacterial | mix | internal | NYC central park soil |

For each dataset a random collection of 100,000 sequenced reads was considered (in fast5 format). Each read was basecalled with bonito v0.5 with High Accuracy model. 5mC methylation inference in CG motifs was performed using Oxford Nanopore's integrated Remora methylation inference algorithm. Then, the full canonical basecalled DNA sequence of each read was taxonomically classified with either kraken2 sequence taxonomic classification method (https://genomebiology.biomedcentral.com/articles/10.1186/s13059-019-1891-0) with the PlusPF database (https://benlangmead.github.io/aws-indexes/k2) or with centrifuge sequence taxonomic classification method (paper: https://genome.cshlp.org/content/early/2016/11/16/gr.210641.116) with the NCBI database (publicly available here: https://benlangmead.github.io/aws-indexes/centrifuge).

For bacterial target class datasets only reads that classified as “bacterial kingdom” (taxid:2) were retained. For non-bacterial target datasets only reads that classified with a taxonomy id equal to the respective dataset's organism were retained. A subcollection of 150,000 bacterial-classified reads and 150,000 non-bacterial classified reads were further retained (equal random selection from respective target datasets). Each read was assigned a class label (bacterial/vertebrate/plant) based on the taxonomical classification results. Each read was further assigned an organism label (bacterial/specific organism based on taxonomical classification results and the target dataset's organism).

For each retained read, the first N canonical basepairs of sequence were considered, where N was drawn at random from a normal distribution with a mean of 500 and a standard deviation of 80 (representing the expected length of the portion of the DNA molecule translocated through the nanopore within the first 1 second of sequencing, assuming ˜450 bp/s translocation speed).

For the first N canonical base pairs of each read, the 5mC methylation inference results within the CG motifs of the sequence were considered. The respective methylation profiles were pre-processed for pAMS and aAMS classifiers as discussed above. For both pAMS and aAMS classifiers, the considered dataset of 300,000 reads' pre-processed methylation profiles and associated per-read class and organisms labels was divided at random into a 90%/10% split for training/validation purposes. Both pAMS and aAMS classifiers were trained with recommended program setups for a binary classification task of separating bacterial vs non-bacterial reads based on the input pre-processed methylation profiles with the previously mentioned 90% (training part) of the pre-processed 300,000 reads input.

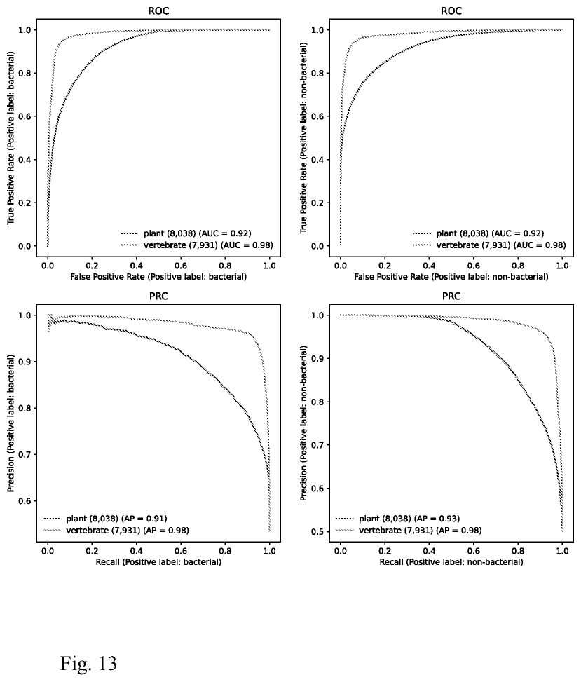

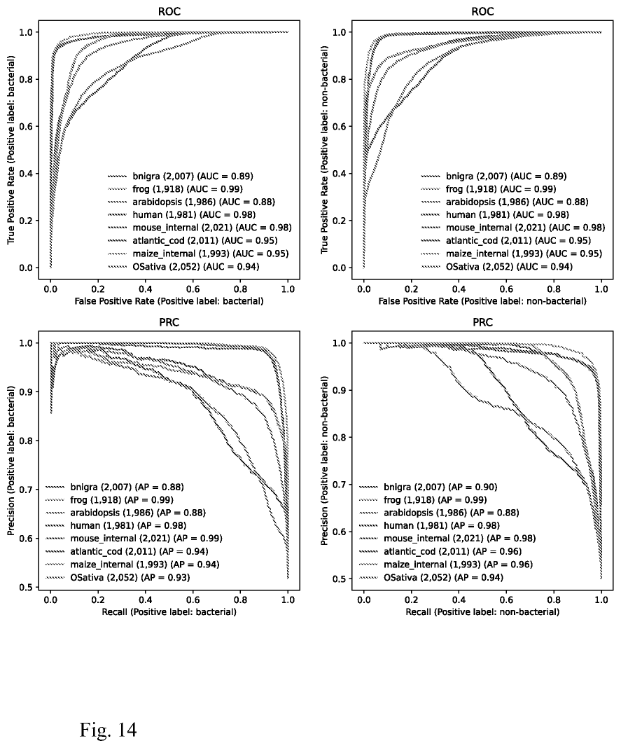

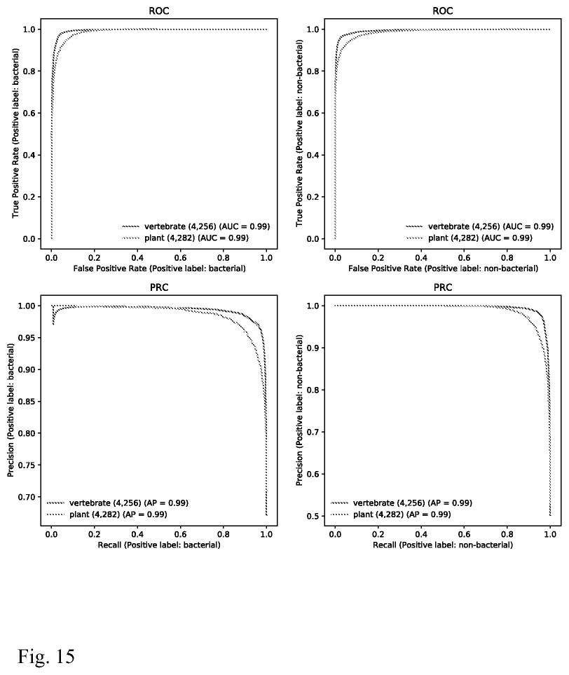

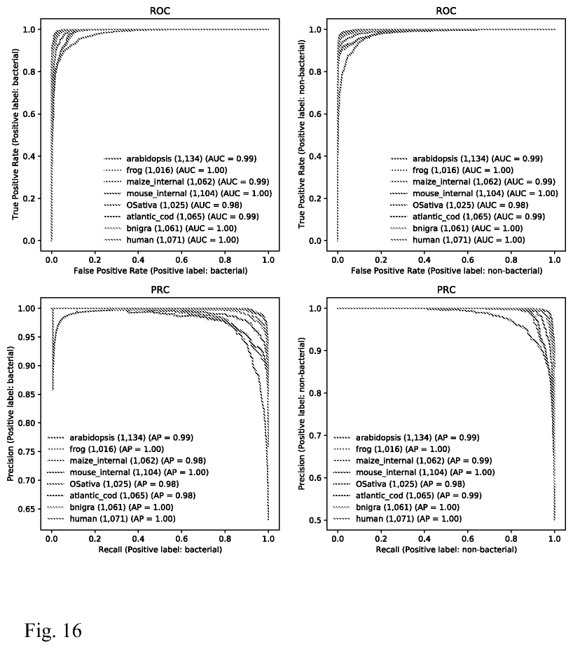

Classifier performance for both the class label and organism level dataset subsets in terms of precision, recall, and AUC metrics is shown in FIG. 13, FIG. 14, FIG. 15, and FIG. 16.

FIG. 13 and FIG. 14 show the results for the pAMS embodiment, in which the polymer is classified as belonging to one of the set of classes based on the modification information 20 only. FIG. 13 shows results for class label classification and FIG. 14 shows results for organism level datasets.

FIG. 15 and FIG. 16 show the results for the aAMS embodiment, in which the polymer is classified as belonging to one of the set of classes based on the modification information 20 and the sequence information 30. FIG. 15 shows results for class label classification and FIG. 16 shows results for organism level datasets.

Further testing of the pAMS embodiment was conducted to determine its effect on read lengths of target and background classes, and thereby the ability to enrich/deplete polymers from the target/background classes. This testing comprised “playback simulation”. For the playback simulation, real-time measurements of a nanopore sequencing experiment are “played back” via the nanopore sequencing controlling firmware. Control of the playback experiment can be taken via the previously outlined pAMS embodiment. In this testing, a “recording” of a full-length WGS nanopore sequencing experiment was considered with input molecules comprised of ˜95% zymo bacterial community DNA and ˜5% human HG002 cell line DNA. The input DNA mixture was prepared following the standard ONT ligation library preparation workflow with a ligation sequencing kit SQK-LSK110 and the sequencing was performed on GridION device with a MinION flowcell with a pore versioned 9.4.1.

The main difference between a playback simulation and a real live sequencing experiment is that during the playback simulation a reject signal sent for a particular nanopore would not result in a sequenced molecules being ejected from the pore (for another molecule to take its place down the line). Instead, the firmware saves the information about the sequenced stretch of the molecule in question prior to the reject signal being sent, and considers any signal further measured in this pore to be coming from a separate molecule.

Playback simulation allows the effectiveness of the present method to be observed. The effectiveness is measured not in the change of the yield of on-target vs off-target polymer molecules sequenced. Rather effectiveness is measured by the length distribution changes of the on-target vs off-target molecules as well as the number of reads being on-target vs off-target. This is because off-target reads are effectively split into a larger collection of shorter versions of their full-length selves.

Playback simulation was performed both with and without the use of the present method interacting with the sequencing process. Two replicates of the sequencing playback were performed with distinct collections of 128 channels (1-128, 256-384) being controlled with the present method during the first hour of sequencing (time limitation to the first hour of sequencing was chosen for convenience). The reported sequenced results (fast5 files) in each instance were further basecalled with guppy 6.1.3 basecalling algorithm with High Accuracy model and classified with kraken2 taxonomic classification software as to infer bacterial and human reads from the reported data.

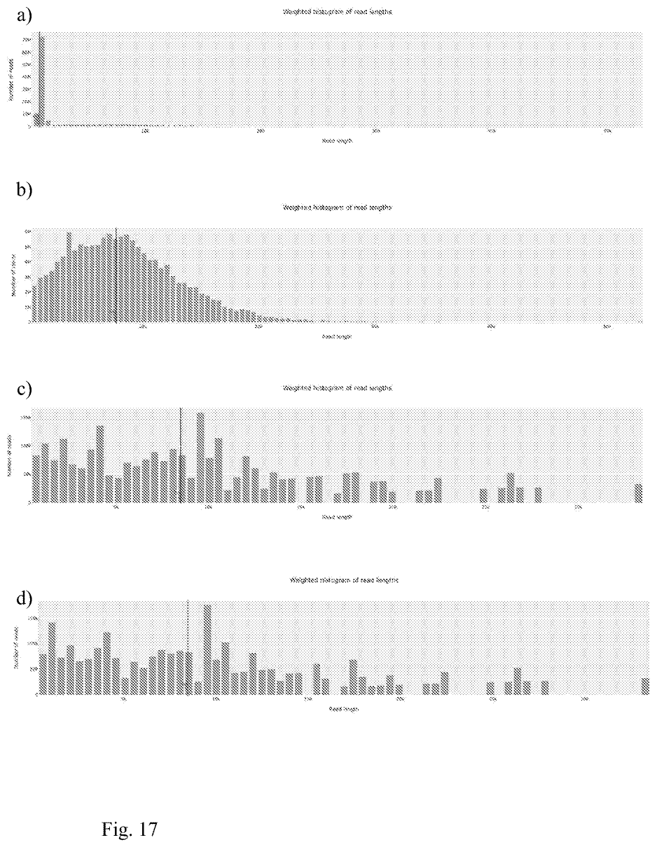

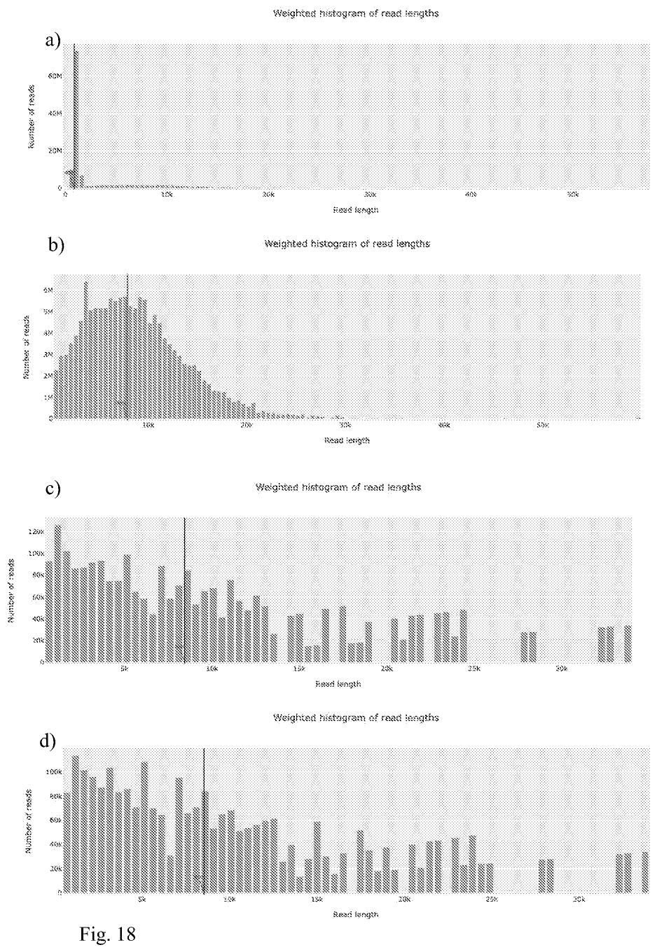

Basecalled and successfully classified reads were further split into bacterial vs human subsets and further processed with NanoPlot software (https://academic.oup.com/bioinformatics/article/34/15/2666/4934939) to visualize read length distributions and other summary statistics. Comparisons are provided in FIG. 17 and FIG. 18 and the tables below for bacterial vs human reads in experiments controlled with and without the present method.

FIG. 17, Table 2, and Table 3 show the results for channels 1-128. FIG. 17a) shows bacterial read lengths with depletion of polymers classified as bacterial using the present method, and FIG. 17b) shows bacterial read lengths without using the present method. The same results as FIG. 17a) and FIG. 17b) are shown in Table 2.

TABLE 2
Channels 1-128
Bacterial reads

| General | Bacterial depletion intervention | Uninterrupted |
|---|---|---|
| summary | (using present method) | sequencing |
| Mean read length | 987.1 | 4,338.7 |
| Median read length | 703.0 | 3,054.0 |
| Number of reads | 132,309.0 | 32,068.0 |
| Read length N50 | 828.0 | 7,709.0 |
| STDEV read length | 1,525.9 | 4,163.8 |
| Total bases | 130,607,031.0 | 139,134,017.0 |

FIG. 17c) shows human read lengths with depletion of polymers classified as bacterial using the present method, and FIG. 17d) shows human read lengths without using the present method. The same results as FIG. 17c) and FIG. 17d) are shown in Table 3.

Human reads

| General | Bacterial depletion intervention | Uninterrupted |
|---|---|---|
| summary | (using present method) | sequencing |
| Mean read length | 3,403.9 | 3,700.5 |
| Median read length | 1,246.0 | 1,702.0 |
| Number of reads | 799.0 | 739.0 |
| Read length N50 | 8,484.0 | 8,516.0 |
| STDEV read length | 4,634.0 | 4,769.1 |
| Total bases | 2,719,712.0 | 2,734,633.0 |

FIG. 18, Table 4, and Table 5 show the results for channels 256-384. FIG. 18a) shows bacterial read lengths with depletion of polymers classified as bacterial using the present method, and FIG. 18b) shows bacterial read lengths without using the present method. The same results as FIG. 18a) and FIG. 18b) are shown in Table 4.

TABLE 4
Channels 256-384
Bacterial reads

| General | Bacterial depletion intervention | Uninterrupted |
|---|---|---|
| summary | (using present method) | sequencing |
| Mean read length | 1,018.6 | 4,482.9 |
| Median read length | 735.0 | 3,220.0 |
| Number of reads | 131,692.0 | 32,093.0 |
| Read length N50 | 878.0 | 7,894.0 |
| STDEV read length | 1,589.5 | 4,294.5 |
| Total bases | 134,139,905.0 | 143,869,675.0 |

FIG. 18c) shows human read lengths with depletion of polymers classified as bacterial using the present method, and FIG. 18d) shows human read lengths without using the present method. The same results as FIG. 18c) and FIG. 18d) are shown in Table 5.

TABLE 5
Human reads

| General | Bacterial depletion intervention | Uninterrupted |
|---|---|---|
| summary | (using present method) | sequencing |
| Mean read length | 3,466.3 | 3,672.6 |
| Median read length | 1,399.0 | 1,615.0 |
| Number of reads | 779.0 | 751.0 |
| Read length N50 | 8,436.0 | 8,533.0 |
| STDEV read length | 4,904.5 | 5,016.1 |
| Total bases | 2,700,252.0 | 2,758,130.0 |

In the performed playback simulation experiment, the average time required for the AMS classifier to preprocess the data and make a classification resulting in a continue/reject for all input molecules/channels was: ˜0.1 seconds for 128 nanopore channel data input. AMS computation performance of ˜0.18 seconds for 256 nanopore channel input, and ˜0.3 seconds for 512 nanopore channel input was also observed.

1. A method of controlling a biochemical analysis system for analysing polymers that comprise a sequence of polymer units, wherein the biochemical analysis system comprises at least one sensor element that comprises a nanopore, and the biochemical analysis system is operable to take successive measurements of a polymer from the sensor element during translocation of the polymer with respect to the nanopore of the sensor element, the method comprising:
when a polymer has partially translocated through the nanopore, analysing the measurements taken from the polymer during the partial translocation thereof to determine modification information in respect of a portion of the sequence of the polymer units, the modification information representing a sequence of estimates of modification statuses of subject polymer units of the portion of modification with respect to at least one canonical type of polymer unit;
classifying the polymer as belonging to one of a set of classes based on the modification information; and
operating the biochemical analysis system to reject the polymer or continue taking measurements from the polymer based on the class to which the polymer unit is classified as belonging.

2. The method of claim 1, wherein the estimates of modification statuses comprise scores in respect of the subject polymer units.

3. The method of claim 1 or claim 2, further comprising determining sequence information representing estimates of the identities of the polymer units of the portion of the sequence of polymer units.

4. The method of claim 3, wherein the estimates of the identities of the polymer units comprise scores in respect of each of a set of types of polymer units.

5. The method of claim 3 or 4, wherein
the estimates of the identities of the polymer units are estimates in respect of a set comprising canonical types of polymer units, and
determining the modification information comprises analysing the sequence information to determine the modification information.

6. The method of claim 3 or 4, wherein the estimates of the identities of the polymer units are estimates in respect of a set comprising canonical types of polymer units and one or more modified forms of at least one canonical type of the polymer units, wherein the modification information comprises the estimates in respect of the modified forms of the at least one canonical type of the polymer units.

7. The method of any of claims 3 to 6, wherein the polymer is classified as belonging to one of the set of classes based on the modification information and the sequence information.

8. The method of any one of claims 1 to 6, wherein the polymer is classified as belonging to one of the set of classes based on the modification information only.

9. The method of any preceding claim, wherein the polymer derives from an organism, and wherein the classes of the set of classes are taxonomic domains or kingdoms.

10. The method claim 9, wherein a first class of the set of classes is bacterial organisms or a type of bacterial organism, and a second class of the set of classes is eukaryotic organisms or a type of eukaryotic organism.

11. The method of any preceding claim, wherein at least one class of the set of classes represents a target sequence, and the step operating the biochemical analysis system to reject the polymer or continue taking measurements from the polymer comprises operating the biochemical analysis system to continue taking measurements when the class to which the polymer unit is classified as belonging by the machine learning classifier being said at least one class representing a target sequence.

12. The method of any preceding claim, wherein at least one class of the set of classes represents a background sequence, and the step operating the biochemical analysis system to reject the polymer or continue taking measurements from the polymer comprises operating the biochemical analysis system to reject the polymer when the class to which the polymer unit is classified as belonging by the machine learning classifier being said at least one class representing a background sequence.

13. The method of any preceding claim, wherein the polymer is a polynucleotide, and the polymer units are nucleotides.

14. The method of any preceding claim, wherein the modification statuses are methylation statuses.

15. The method of claim 14, wherein:
the subject polymer units are cytosine nucleotides and the methylation statuses are statuses of methylation to at least one of 5-methyl-cytosine or 5-hydroxymethyl-cytosine; and/or
the subject polymer units are adenosine nucleotides and the methylation statuses are statuses of methylation to 6-methyl-adenine.

16. The method of any preceding claim, wherein the modification statuses are oxidation statuses.

17. The method of any preceding claim, wherein the subject polymer units comprise polymer units forming part of a predetermined motif of polymer units.

18. The method of claim 17, wherein the predetermined motif of polymer units is a cytosine nucleotide followed by a guanine nucleotide in the sequence of nucleotides along a 5′→3′ direction.

19. The method of any preceding claim, wherein the at least one sensor element is operable to eject a polymer that is translocating through the nanopore, wherein operating the biochemical analysis system to reject the polymer comprises operating the sensor element to eject the polymer from the nanopore and accept a further polymer in the nanopore.

20. The method of claim 21, wherein the at least one sensor element is operable to eject a polymer that is translocating through the nanopore by application of an ejection bias voltage sufficient to eject the polymer, wherein operating the sensor element to eject the polymer from the nanopore is performed by applying an ejection bias voltage.

21. The method of any preceding claim, wherein classifying the polymer as belonging to one of the set of classes comprises inputting the modification information into a machine learning classifier that classifies the polymer as belonging to one of the set of classes based on the modification information.

22. The method of claim 21, wherein the machine learning classifier comprises a neural network.

23. The method of claim 21 or 22, wherein the machine learning classifier is trained using modification information in respect of plural classes.

24. A computer program comprising instructions which, when the program is executed by a computer, cause the computer to carry out a method according to any one of the preceding claims.

25. A computer storage medium storing a computer program according to claim 24.

26. A biochemical analysis system for analysing polymers that comprise a sequence of polymer units, the biochemical analysis system comprising at least one sensor element that comprises a nanopore, wherein the biochemical analysis system is operable to take successive measurements of a polymer from the sensor element during translocation of the polymer with respect to the nanopore of the sensor element;
wherein the biochemical analysis system is configured to perform the method of any of claims 1 to 23.

27. The biochemical analysis system of claim 24, wherein the biochemical analysis system is a portable biochemical analysis system.
